(19) United States
(12) Patent Application Publication
(10) Pub. No.: US 2025/0154149 A1
(43) Pub. Date: May 15, 2025

(54) PHARMACEUTICAL COMPOSITION AND METHOD FOR PREPARING ACTIVE INGREDIENT COMPOUND THEREOF

(71) Applicant: Betta Pharmaceuticals Co., Ltd., Zhejiang (CN)

(72) Inventors: Xiaofeng XU, Beijing (CN); Xizhen SONG, Beijing (CN); Xintao ZHAO, Zhejiang (CN); Yan XU, Beijing (CN); Liang CHEN, Zhejiang (CN); Hongfei RONG, Beijing (CN); Zongquan LI, Beijing (CN); Xiangyong LIU, Beijing (CN); Lieming DING, Zhejiang (CN); Jiabing WANG, Beijing (CN)

(73) Assignee: Betta Pharmaceuticals Co., Ltd., Zhejiang (CN)

(21) Appl. No.: 18/839,000

(22) PCT Filed: Feb. 14, 2023
(86) PCT No.: PCT/CN2023/075802

§ 371 Date: Aug. 15, 2024

(30) Foreign Application Priority Data
Feb. 16, 2022 (WO) PCT/CN2022/076439

(51) Int. Cl. C07D 471/04; A61K 31/4985; A61P 35/00

(52) U.S. Cl. CPC C07D 471/04; A61K 31/4985; A61P 35/00

(57) ABSTRACT

The present invention relates to a pharmaceutical composition comprising a compound as shown in formula I, and use of the pharmaceutical composition in treating diseases. The present invention also relates to a method for preparing the compound as shown in formula I.

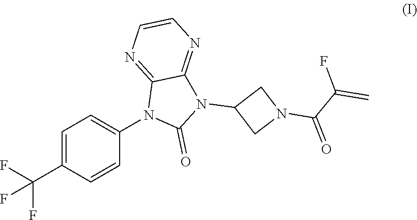 [SMILES: C=C(F)C(=O)N1CC(n2c(=O)n(-c3ccc(C(F)(F)F)cc3)c3nccnc32)C1]

### TECHNICAL FIELD

The present application relates to a pharmaceutical composition comprising a compound represented by Formula I, and use of the pharmaceutical composition in treating disease. The present application also relates to a method for preparing the compound represented by Formula I, and belongs to the field of pharmaceutical chemistry.

### BACKGROUND

In a normal environment, the dynamic balance between cell proliferation and apoptosis maintains the normal size of tissues and organs and the stability of the internal environment. When cell proliferation or apoptosis is out of control, malignant transformation of cells occurs. Hippo signaling pathway is a pathway inhibiting cell growth, consisting of multiple tumor suppressors that regulate the balance between cell proliferation and apoptosis through a series of kinase cascade reactions. The Hippo signaling pathway plays a key role in early embryonic development, organ size and regeneration, and the like.

The Hippo pathway was initially discovered in Drosophila as an important developmental pathway controlling organ size, and subsequently also discovered in mammals. In mammals, the Hippo pathway can be divided into three categories: upstream regulatory components (NF2/Merlin, GPCRS, etc.), core kinase cascades (MST1/2, LATS1/2, and regulatory proteins SAV1 and MOB), and downstream effector molecules (YAP/TAZ). Tumor suppressor protein neurofibromatosis type 2 antigen (NF2/Merlin) or other upstream regulatory signals activate MST1/2 kinase and scaffold protein SAV1. Activated MST1/2 promotes phosphorylation of LATS1/2 and MOB. Phosphorylated LATS1/2 can further phosphorylate YAP/TAZ to realize the regulation of the Hippo signaling pathway. The connections of phosphorylated YAP/TAZ to 14-3-3 mediating cytoplasmic retention and β-TrCP mediating the degradation of proteasome are eventually degraded.

Unphosphorylated YAP/TAZ in the cytoplasm crosses the nuclear membrane into the nucleus and binds to TEADs proteins to form a transcription activation complex, thereby regulating transcription of downstream genes. Many cytokines, including connective tissue growth factor (CTGF), cysteine-rich angiogenic inducer 61 (CYR61), ankyrin repeat domain 1 (ANKRD1), baculoviral IAP repeat containing 5 (BIRC5), brain-derived neurotrophic factor, fibroblast growth factor 1, and the like, are all downstream target genes regulated by YAP/TAZ-TEAD. CTGF, as a direct target gene of YAP/TAZ-TEAD, may promote cell proliferation and cell growth.

The human YAP gene is located on chromosome 11q13 and is widely expressed in various tissues except peripheral blood cells. YAP comprises a plurality of structural domains and specific amino acid sequences, including a TEAD-binding region, a WW structural domain, a proline-rich N-terminal structural domain, a PDZ-binding motif at the C-terminal, a SH3-binding motif, a coiled-coil structural domain, and a transcription activation structural domain. The WW structural domain specifically recognizes the PPXY motif to mediate the formation of transcription complex. TAZ is a homologous protein of YAP with only one WW structural domain.

The TEAD family is the most important transcription factor for YAP and TAZ. Point mutations at key positions of TEAD, especially those associated with the YAP and TEAD binding domain, significantly inhibit the expression and function of YAP-induced genes. The human TEAD family transcription factors include four members, TEAD1, TEAD2, TEAD3, and TEAD4, with high homology. TEADs include a TEA-binding structural domain at the N-terminal, which serves as a site for binding to DNA transcription promoters, and a YAP/TAZ-binding structural domain at the C-terminal. The N-terminal structural domain of YAP/TAZ wraps around the C-terminal structural domain of TEAD to form a spherical structure. The binding region of YAP/TAZ and TEAD is divided into three interfaces. Interface 1 is mediated by seven intermolecular hydrogen bonds between the peptide backbones of YAP β1 and TEAD β7, forming an antiparallel β-fold. Interface 2 is generated by YAP α1 helices close to the groove formed by TEAD α3 and TEAD α4. At interface 3, the Q-ring of YAP interacts with the deep pockets formed by β4, β11, β12, α1 and α4 of TEAD.

Typically, YAP/TAZ is only induced in specific tissues and under specific conditions (e.g. development, wound healing, and the like). It is expressed at low levels in other tissues. Mutations in Hippo pathway components trigger the hyperactivation of YAP/TAZ, leading to the proliferation of normal cells. Studies have shown that after dysregulation of the Hippo pathway, hyperactivation of YAP/TAZ is prevalent in cancer such as lung cancer, liver cancer, pancreatic cancer, breast cancer, and the like.

In cancer stem cells of various solid tumors, YAP/TAZ can promote cancer stem cell survival and is closely related to cancer cell metastasis and drug resistance, promoting the development and progression of various tumors. During chemotherapy, anti-microtubule medicaments, antimetabolites and DNA damaging agents can affect the Hippo signaling pathway, leading to the activation and transcription of YAP/TAZ, resulting in drug resistance. Hyperactivation of YAP/TAZ can cause high expression of several medicament transporter proteins, which can transfer medicament to the extracellular compartment, leading to the up-regulation of anti-apoptotic proteins such as Bcl and survivin, thereby inhibiting apoptosis. Many studies have shown that PD-L1 is a direct transcription target of YAP/TAZ. Activated YAP/TAZ can increase the expression of PD-L1. The activated YAP/TAZ can also induce the expression of cytokines IL-6, CSF1-3, TNFA, IL-3, CXCL1/2, CCL2, and the like to promote the recruitment and polarization of myeloid-derived suppressor cells (MDSCs), inactivating T cells or inducing the apoptosis of T cells. More studies have shown that the dismissal of the down-regulation of the Hippo pathway causes activation of YAP/TAZ, which is a major mechanism for a variety of targeted drug resistance. YAP/TAZ-activated transcription can overcome EGFR resistance by multiple mechanisms. For example, high expression of AXL mediates the resistance to EGFR inhibitor in NSCLC; inhibition of pro-apoptotic protein BMF mediates the resistance to EGFR/MEK inhibitors; and activation of the PI3K/AKT signaling pathway to evade targeted therapy. YAP-activated transcription can also mediate the resistance to BRAF, KRAS, and MAPK inhibitors. The activation of YAP/TAZ is not only associated with drug resistance, studies have shown that YAP gene amplification is also associated with the recurrence of colon and pancreatic cancer.

Thus, the Hippo pathway plays an important role in controlling the morphology of tissues and organs. It is associated with many aspects of tumorigenesis, including cell proliferation, differentiation, apoptosis, tissue regeneration, cancer metastasis, and cancer therapy resistance. Abnormal regulation of the Hippo pathway can lead to high expression and activation of YAP/TAZ in the cytoplasm and nucleus, thereby inducing tumor development and metastasis, and even generating drug resistance. Disruption of YAP/TAZ-TEAD interaction can eliminate the carcinogenic properties of YAP/TAZ. Thus, a theoretical basis is provided for the treatment of these cancers by protein-protein interaction inhibitors of YAP/TAZ and TEAD.

The present application has found that 1-[1-(2-fluoroacryloyl) azetidin-3-yl]-3-(4-trifluoromethylphenyl)-1,3-dihydro-2H-imidazo[4,5-b]pyrazin-2-one has excellent cytostatic activity, good pharmacokinetic characteristics, and excellent antitumor activity, showing potential for use in the treatment of a disease mediated by the abnormality of Hippo pathway. The present application also discovered that advantageous crystalline solid form of 1-[1-(2-fluoroacryloyl) azetidin-3-yl]-3-(4-trifluoromethylphenyl)-1,3-dihydro-2H-imidazo[4,5-b]pyrazin-2-one has good physicochemical stability, suitable solubility, excellent pharmacokinetic properties, and a suitable crystallization process, which is favorable for medicament development.

### SUMMARY

The present application relates to a pharmaceutical composition comprising 5 wt. % to 90 wt. % of a compound represented by Formula I, or a crystalline form thereof, or a mixture thereof,

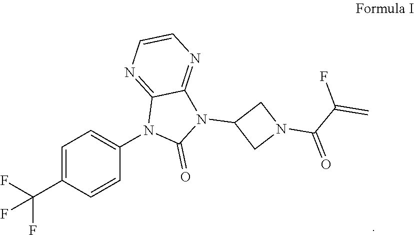 [SMILES: C=C(F)C(=O)N1CC(n2c(=O)n(-c3ccc(C(F)(F)F)cc3)c3nccnc32)C1]

As noted above, the term “compound represented by Formula I” denotes 1-[1-(2-fluoroacryloyl) azetidin-3-yl]-3-(4-trifluoromethylphenyl)-1,3-dihydro-2H-imidazo[4,5-b]pyrazin-2-one.

In some embodiments, the crystalline form of the compound represented by Formula I is a crystal form A.

In some embodiments, the present application also provides the crystal form A of the compound represented by Formula I.

In some embodiments, the present application also provides use of the pharmaceutical composition in the manufacture of medicaments.

In some embodiments, the present application also provides a method for treating a disease, comprising administering a therapeutically effective amount of the pharmaceutical composition to a subject to be treated.

In some embodiments, the present application also provides a preparation method of the compound represented by Formula I or the crystal form A thereof.

### DETAILED DESCRIPTION

Although preferred embodiments of the present application are shown and described herein, such embodiments are provided by way of example only and are not intended to limit the scope of the present application. Various alternatives to the described embodiments of the present application may be used in the practice of the present application.

### Crystalline Form

In some embodiments, the present application provides a pharmaceutical composition comprising 5 wt. % to 90 wt. % of a compound represented by Formula I below,

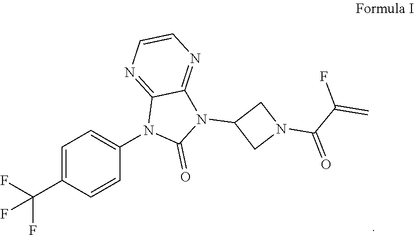 [SMILES: C=C(F)C(=O)N1CC(n2c(=O)n(-c3ccc(C(F)(F)F)cc3)c3nccnc32)C1]

In some embodiments, the present application provides a crystalline form of the compound represented by Formula I.

In some embodiments, the pharmaceutical composition comprises 5 wt. % to 90 wt. % of the crystalline form of the compound represented by Formula I.

In some embodiments, the crystalline form of the compound represented by Formula I is a crystal form A, and the crystal form A has an X-ray powder diffraction pattern comprising characteristic peaks at 2θ values of 8.2°±0.2°, 15.4°±0.2°, and 18.2°±0.2°.

In some embodiments, the crystalline form of the compound represented by Formula I is a crystal form A, and the crystal form A has an X-ray powder diffraction pattern comprising characteristic peaks at 2θ values of 6.4°±0.2°, 7.7°±0.2°, 8.2°±0.2°, 12.8°±0.2°, 15.4°±0.2°, and 18.2°±0.2°.

In some embodiments, the crystalline form of the compound represented by Formula I is a crystal form A, and the crystal form A has an X-ray powder diffraction pattern comprising characteristic peaks at 2θ values of 6.4°±0.2°, 7.7°±0.2°, 8.2°±0.2°, 12.8°±0.2°, 15.4°±0.2°, 18.2°±0.2°, and 20.3°±0.2°.

In some embodiments, the crystalline form of the compound represented by Formula I is a crystal form A, and the crystal form A has an X-ray powder diffraction pattern comprising characteristic peaks at 2θ values of 6.4°±0.2°, 7.7°±0.2°, 8.2°±0.2°, 12.8°±0.2°, 15.4°±0.2°, 18.2°±0.2°, 20.3°±0.2°, and 21.6°±0.2°.

In some embodiments, the crystalline form of the compound represented by Formula I is a crystal form A, and the crystal form A has an X-ray powder diffraction pattern comprising characteristic peaks at 2θ values of 6.4°±0.2°, 7.7°±0.2°, 8.2°±0.2°, 12.8°±0.2°, 15.4°±0.2°, 18.2°±0.2°, 20.3°±0.2°, 21.6°±0.2°, 23.3°±0.2° and 25.6°±0.2°.

In some embodiments, the crystal form A has an X-ray powder diffraction pattern substantially as shown in FIG. 1.

In some embodiments, the crystal form A has a thermogravimetric analysis (TGA) pattern substantially as shown in FIG. 2, with a weight loss of about 0.2% before 200° C.

In some embodiments, the crystal form A has a differential scanning calorimetry (DSC) pattern substantially as shown in FIG. 3, with an onset melting temperature of about 169° C. and a peak temperature of about 170° C.

The crystal form A is characterized by single crystal analysis, and the single crystal of the crystal form A has a crystal cell stacking projection substantially as shown in FIG. 4, with cell parameters shown as below:
the crystal belongs to the triclinic crystal system, with a space group of P-1, and the following cell parameters:
a=10.1991(1)Å,
b=12.9256(1)Å,
c=14.5960(2)Å;
α=107.94(1)°,
β=91.191(1)°,
γ=109.437(1)°;
cell volume V=1709.87(3)Å³,
number of asymmetric units in the cell Z=2.

As the final reliability factors, R₁=0.0503, wR₂=0.1431, and S=1.025. The final stoichiometric formula of the asymmetric unit is determined to be 2(C₁₈H₁₃F₄N₅O₂), the molecular weight of the individual molecule is calculated to be 407.33, and the density of the crystal is calculated to be 1.582 g/cm³.

The ellipsoidal graph of the stereostructure of the asymmetric unit for a single crystal of the crystal form A is shown in FIG. 5.

All crystal forms of the present application are substantially pure.

All the X-ray powder diffraction patterns were measured using the Ka spectrum of Cu target, unless otherwise stated.

The experimental temperatures of the present application are all room temperature, unless otherwise stated.

The term “substantially pure” as used herein means that the content by weight of the crystal form is not less than 85%, preferably not less than 95%, more preferably not less than 98%.

It is to be noted that the numerical values and numerical ranges referred to in the present application should not be narrowly construed as numerical values or numerical ranges per se, and those skilled in the art should understand that they may fluctuate around the specific numerical values according to the specific technical environment without departing from the spirit and principles of the present application. In the present application, such fluctuating ranges as may be foreseen by those skilled in the art are mostly expressed by the terms “about” or “substantially”.

The term “crystalline form” as used in the present application refers to crystal forms having the same chemical composition but different spatial arrangements of molecules, atoms and/or ions forming the crystals, including anhydrous crystal forms, hydrates and solvates. The terms “polymorph”, “crystal form”, “crystalline form” and “polycrystalline form” herein can be used interchangeably, all of which denote the solid form of the compound represented by Formula I, and are different from the amorphous form of the compound represented by Formula I. The term “amorphous form” refers to a non-crystalline solid form. The polymorphs of the compound may have different chemical and/or physical properties, including, but not limited to, for example, stability, solubility, dissolution rate, optical properties, melting point, mechanical properties, and/or density, and the like. These properties can affect the processing and/or manufacturing of the bulk drug, as well as the stability, dissolution and/or bioavailability of the medicament. Accordingly, the polymorphism can affect at least one property of the medicament, including, but not limited to, quality, safety, and/or efficacy. In the absence of a precise definition, the compound represented by Formula I includes amorphous form, any crystalline form, a mixture of any two or more crystalline forms, and a mixture of any one or more crystalline forms with amorphous form.

Polymorph of the molecule can be obtained by many methods known in the art. Such methods include, but are not limited to, melt recrystallization, melt cooling, solvent recrystallization, desolventization, rapid evaporation, rapid cooling, slow cooling, vapor diffusion, and sublimation. The polymorph can be detected, identified, classified and characterized using well known techniques including, but not limited to, differential scanning calorimetry (DSC), thermogravimetry (TGA), X-ray powder diffraction (XRPD), single crystal X-ray diffraction, solid-state nuclear magnetic resonance (NMR), infrared (IR) spectroscopy, raman spectroscopy, and hot stage optical microscopy.

In the present application, the term “substantially” used in “having an X-ray powder diffraction pattern substantially as shown in FIG. 1” indicates that the exact positions of the peaks in the accompanying drawings should not be interpreted as absolute values. Since it is known to those skilled in the art that the 2θ values of the X-ray powder diffraction pattern may be subject to error due to different measurement conditions (e.g., equipments and instruments used) and different samples (e.g., different batches of samples), and the error in the measurement of the diffraction angle of the X-ray powder diffraction pattern can be 5% or less. A difference of =0.2° of a given value is generally considered to be appropriate. It should also be understood that the relative intensity of the peaks may fluctuate with experimental conditions and sample preparation, such as the preferential orientation of particles in the sample. The use of automatic or fixed divergence slits can also affect the calculation of relative intensities. The intensities shown in the XRD curves included herein are only exemplary and cannot be used for absolute comparisons, and any crystalline form having a powder diffraction pattern substantially the same as those disclosed herein is within the scope of protection of the present application.

It will be understood by those skilled in the art that data measured by DSC may vary somewhat due to variations in sample batch, sample purity, sample preparation and measurement conditions (e.g., heating rate), wherein a difference of +5° C. from a given value is usually acceptable (and is still considered to be characteristic of the particular crystal form described herein). Accordingly, the endothermic pattern cited in the present application should not be taken as absolute values, and such errors should be taken into account when interpreting the DSC data.

In the TGA test, without being limited by any particular theory, the weight loss corresponds to the loss of trace residual solvent or water. The data measured by TGA may vary somewhat due to variations in sample batch, sample purity, sample residual solvent content, sample preparation and measurement conditions (e.g., heating rate). Accordingly, the thermogravimetric pattern cited in the present application should not be taken as absolute values, and such errors should be taken into account when interpreting the TGA data.

### Pharmaceutical Composition

In some embodiments, a pharmaceutical composition comprising the compound represented by Formula I or a crystalline form thereof, or a mixture thereof, wherein, the pharmaceutical composition comprises 5 wt. % to 90 wt. % of the compound represented by Formula I according to the present application or the crystalline form thereof, or the mixture thereof.

In some embodiments, the pharmaceutical composition is for use in oral administration dosage form.

In some embodiments, the oral administration dosage form includes tablet, capsule, cachet, pill, granule, oral liquid, suspension, dispersion, emulsion, and powder.

In some embodiments, the pharmaceutical composition comprises 5 wt. % to 70 wt. % of the compound represented by Formula I or a crystalline form thereof, or a mixture thereof. Preferably, the pharmaceutical composition comprises the crystal form A of the compound represented by Formula I.

In some embodiments, the pharmaceutical composition comprises 5 wt. % to 60 wt. % of the compound represented by Formula I or a crystalline form thereof, or a mixture thereof. Preferably, the pharmaceutical composition comprises the crystal form A of the compound represented by Formula I.

In some embodiments, the pharmaceutical composition comprises 10 wt. % to 60 wt. % of the compound represented by Formula I or a crystalline form thereof, or a mixture thereof. Preferably, the pharmaceutical composition comprises the crystal form A of the compound represented by Formula I.

In some embodiments, the pharmaceutical composition comprises one or more pharmaceutically acceptable carriers.

In some embodiments, the pharmaceutically acceptable carrier may comprise one or more of a diluent, a filler, a lubricant, a binder, and a disintegrating agent.

In practice, the compound represented by Formula I of the present application or the crystalline form thereof, or the mixture thereof as the active ingredient is tightly mixed with a pharmaceutical carrier according to conventional medicament mixing techniques to form the pharmaceutical composition. The pharmaceutical carrier may be in various forms, depending on the desired mode of administration, e.g., oral administration or injection (including intravenous injection). Thus, the pharmaceutical composition of the present application may be in the form of individual units suitable for oral administration, for example, capsule, cachet or tablet comprising a predetermined dose of the active component. Further, the pharmaceutical composition of the present application may be in the form of powder, granule, solution, aqueous suspension, non-aqueous liquid, oil-in-water emulsion or water-in-oil emulsion. Alternatively, in addition to the common dosage forms mentioned above, the compound represented by Formula I or the crystalline form thereof, or the mixture thereof may also be administered by means of a controlled release and/or delivery device. The pharmaceutical composition of the present application can be prepared by any pharmaceutical method. Generally, the method comprises the step of associating the active component and a carrier constituting one or more of necessary components. Generally, the pharmaceutical composition is prepared by uniformly and tightly mixing the active component with a liquid carrier or a finely divided solid carrier or a mixture of the liquid carrier and the solid carrier. In addition, the product can be easily prepared to obtain the desired appearance.

The pharmaceutical carrier used in the present application can be, for example, a solid carrier, a liquid carrier or a gaseous carrier. Solid carrier includes lactose, terra alba, sucrose, talc, gelatin, agar, pectin, acacia, magnesium stearate, and stearic acid. Liquid carrier includes syrup, peanut oil, olive oil and water. Gas carrier includes carbon dioxide and nitrogen. In preparing a pharmaceutical oral preparation, any pharmaceutically convenient medium can be used. Water, glycols, oils, alcohols, flavor enhancers, preservatives, colorants, and the like can be used to prepare an oral liquid preparation such as suspension, elixir and solution; and carriers such as starches, sugars, microcrystalline cellulose, diluents, granulating agents, lubricants (e.g., magnesium stearate and microsilica gel), binders (e.g., povidone and gelatin), disintegrating agents (e.g., sodium carboxymethyl starch and sodium cross-linked carboxymethyl cellulose), and the like can be used to prepare an oral solid preparation such as powder, capsule and tablet. Considering the ease of administration, tablet and capsule are preferred for oral preparation, where solid pharmaceutical carrier is used. Optionally, standard aqueous or non-aqueous preparation techniques can be used for the tablet coating.

A tablet containing the compound or pharmaceutical composition of the present application may be formed by compression or molding, optionally together with one or more auxiliary components or adjuvants. Active components in a form of free flowing, such as powder or granule, may be mixed with binder, lubricant, inert diluent, surfactant or dispersing agent, and the mixture may be compressed to prepare a compressed tablet in a suitable machine. A molded tablet may be prepared by wetting the powdered compound or pharmaceutical composition with an inert liquid diluent and then molding in a suitable machine. Preferably, each tablet contains about 0.05 mg to 5 g of active component and each cachet or capsule contains about 0.05 mg to 5 g of active component. For example, a formulation intended for oral administration to humans contains about 0.5 mg to about 5 g of the active component in admixture with suitable and conveniently dosed adjuvant materials, which account for 5% to 95% of the total amount of pharmaceutical composition. The unit dosage form generally contains about 1 mg to about 2 g of the active component, typically 2.5 mg, 5 mg, 10 mg, 25 mg, 50 mg, 100 mg, 200 mg, 300 mg, 400 mg, 500 mg, 600 mg, 800 mg, or 1000 mg.

The pharmaceutical composition provided by the present application suitable for parenteral administration may be prepared in the form of an aqueous solution or suspension by adding the active component to water. Suitable surfactant such as hydroxypropyl cellulose may be included. A dispersed system can also be prepared in glycerol, liquid polyethylene glycol, or a mixture thereof in oil. Further, a preservative may also be included in the pharmaceutical composition of the present application for use in preventing harmful microbial growth.

The present application provides a pharmaceutical composition suitable for injection, including sterile aqueous solution or dispersed system. Further, the above-mentioned pharmaceutical composition can be prepared in the form of sterile powder for the immediate preparation of sterile injectable solution or dispersion. In any event, the final injectable form must be sterile and, for ease of injection, must be readily flowable. Furthermore, the pharmaceutical composition must be stable during preparation and storage. Therefore, preferably, the pharmaceutical composition must be preserved under conditions that are resistant to contamination by microorganisms such as bacteria and fungi. The carrier can be a solvent or dispersion medium, for example, water, ethanol, polyol (e.g., glycerol, propylene glycol, and liquid polyethylene glycol), vegetable oil, and suitable mixtures thereof.

The pharmaceutical composition provided by the present application may be in a form suitable for topical administration, for example, aerosol, emulsion, ointment, lotion, powder, or other similar dosage forms. Further, the pharmaceutical composition provided by the present application can be in a form suitable for use in a transdermal administration equipment. These preparations can be prepared by conventional processing methods using the compound represented by Formula I of the present application, the crystal form thereof, or the mixture thereof. As an example, an emulsion or ointment with the desired consistency is prepared by adding about 5 wt % to 10 wt % of a hydrophilic material and water to the emulsion or ointment.

The pharmaceutical composition provided by the present application may be in the form of rectal administration by using a solid as carrier. Unit-dose suppository is the most typical dosage form. Suitable adjuvants include cocoa butter and other materials commonly used in the art. The suppository may be conveniently prepared by first mixing the pharmaceutical composition with softened or melted adjuvants, and then cooling and moulding.

In addition to the adjuvant components mentioned above, the above preparation formulation may also comprise appropriately one or more additional adjuvant components, such as diluent, buffer, flavoring agent, binder, surfactant, thickener, lubricant and preservative (including antioxidant), and the like. Further, other adjuvants may also include a permeation enhancer that adjusts the isotonic pressure of the medicament and the blood of the intended recipient. The pharmaceutical composition comprising the compound represented by Formula I, the crystal form thereof, or the mixture thereof may be prepared in the form of powder or concentrate.

### Compound Represented by Formula I and the Preparation Method of the Crystal Form a Thereof

Another aspect of the present application provides a preparation method of the compound represented by Formula I.

In some embodiments, the preparation method of the compound represented by Formula I comprises the following steps:

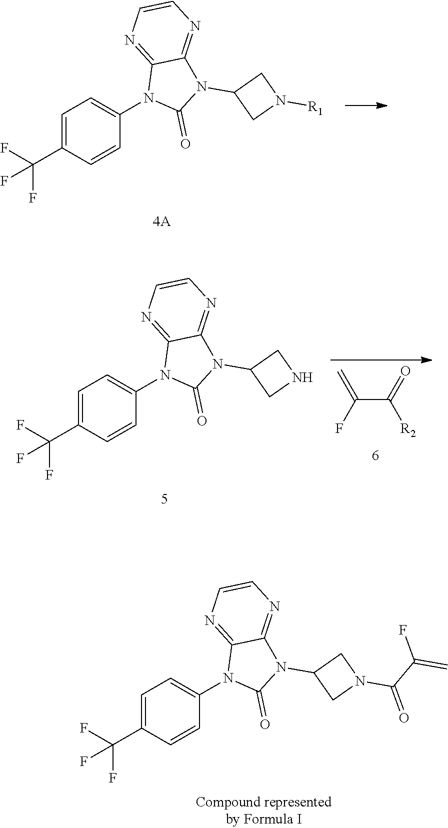

wherein, R₁ is a protecting group selected from the group consisting of —Boc, —Cbz, —Fmoc, trifluoroacetyl, —Trt, —PMB, and —Bn, preferably —Boc;
R₂ is selected from the group consisting of —Cl, imidazolyl and —OR₃; and
R₃ is selected from the group consisting of H, —C₁₋₄ alkyl, phenyl, nitro-substituted phenyl, —C(O)—O—C₁₋₄ alkyl, —C(O)—O-phenyl and —C(O)—O-nitro-substituted phenyl.

Compound 4A is subjected to a deprotection reaction to obtain Compound 5, and Compound 5 and Compound 6 are subjected to a substitution or condensation reaction to obtain the compound represented by Formula I.

In the condensation reaction, the solvent used may be selected from DCM, DMF or a mixed solvent of the above two solvents at any ratio. The condensing agent may be one or more selected from the group consisting of HATU, HBTU, PyBOP, BOP, DCC/HOBT, EDCI/HOBT, EDCI/HOSu, T₃P, CDI, chloroformate, MsCl, TsCl, NsCl, and Boc₂O, preferably HATU. The alkali used in the condensation reaction may be selected from the group consisting of DIPEA and TEA.

The reaction conditions suitable for Compound 4A to remove the protecting groups may be selected depending on the different protecting groups, and the reaction conditions may be acidic conditions, alkaline conditions, or catalytic conditions involving a catalyst. The acid used in the acid conditions may be one or more selected from the group consisting of TFA, H₂SO₄, HCl, and HBF₄, preferably TFA. The catalyst may be selected from the group consisting of H₂—Pd/C and HCOONH₄—Pd/C, preferably H₂—Pd/C. The alkali used in the alkaline conditions may be selected from the group consisting of diethylamine and piperidine, preferably diethylamine.

In some embodiments, in the preparation method of the compound represented by Formula I, the preparation method of Compound 4A may include method 1 or method 2 below:

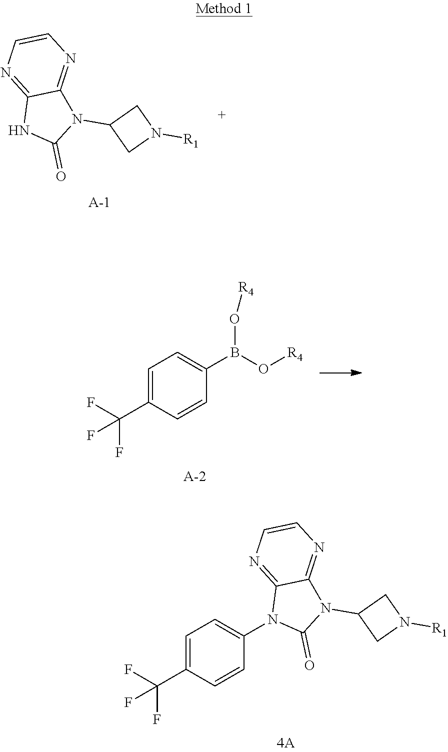

wherein, R₁ is a protecting group that can be one selected from the group consisting of —Boc, —Cbz, —Fmoc, trifluoroacetyl, —Trt, —PMB, and —Bn, preferably —Boc; and

R₄ is selected from the group consisting of H and C₁₋₄ alkyl, or two R₄ and linked O atoms thereof together form 5-membered heterocycle containing two oxygen atoms and one boron atom, wherein the 5-membered heterocycle is optionally substituted with one or more C₁₋₄ alkyl.

Compound A-1 and Compound A-2 are subjected to a Chan-Lam coupling reaction under alkaline conditions (e.g., DIPEA or TEA) to obtain Compound 4A.

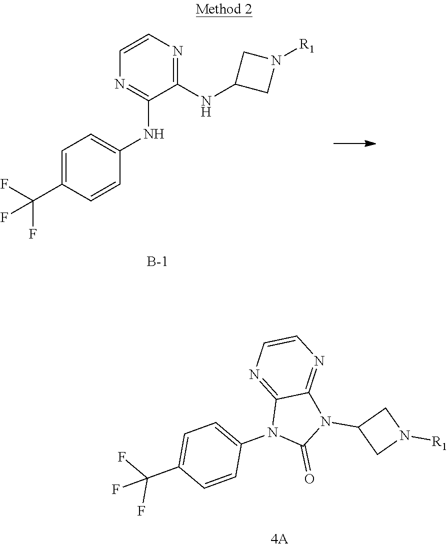

wherein, R₁ is a protecting group that can be one selected from the group consisting of —Boc, —Cbz, —Fmoc, trifluoroacetyl, —Trt, —PMB, and —Bn, preferably —Boc; and

Compound B-1 was subjected to a ring-closing reaction with CDI, triphosgene or p-nitrophenyl chloroformate under alkaline conditions to obtain Compound 4A.

The solvent in the ring-closing reaction is selected from the group consisting of DMF, NMP and acetonitrile, and the alkali is selected from the group consisting of TEA and DIPEA.

In some embodiments, in the preparation method of the compound represented by Formula I, the preparation method of Compound B-1 may be prepared by the following steps:

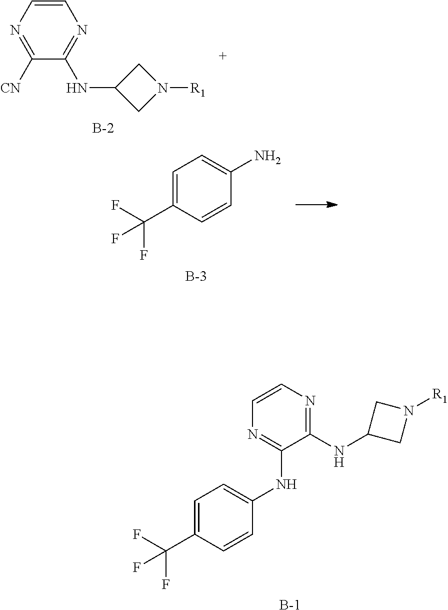

wherein, R₁ is a protecting group that can be one selected from the group consisting of —Boc, —Cbz, —Fmoc, trifluoroacetyl, —Trt, —PMB, and —Bn, preferably —Boc; and

Compound B-2 and Compound B-3 are subjected to a catalytic coupling reaction in the presence of a solvent, an alkali, a catalyst and a ligand to obtain compound B-1.

The solvent may be one or more selected from the group consisting of 1,4-dioxane, DMF, DME, 2-MeTHE, t-BuOH, n-BuOH, toluene/water, and toluene; the alkali may be one or more selected from the group consisting of t-BuONa, K₂CO₃, Cs₂CO₃, and K₃PO₄; the ligand may be one or more selected from the group consisting of BINAP, BrettPhos, DavePhos, Dppf, P(t-Bu)₃, HBF₄, RuPhos, SPhos, XPhos, and XantPhos; and the catalyst may be selected from the group consisting of Pd₂(dba)₃ and Pd(OAc)₂.

In some embodiments, in the preparation method of the compound represented by Formula I, Compound B-2 may be prepared by method 3 or method 4 below:

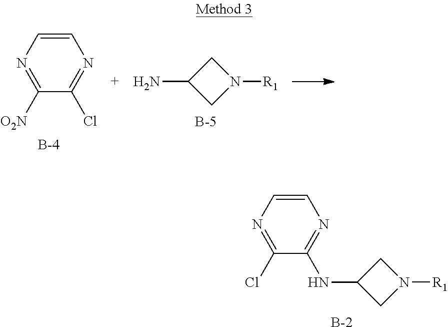

wherein, R₁ is a protecting group that can be one selected from the group consisting of —Boc, —Cbz, —Fmoc, trifluoroacetyl, —Trt, —PMB, and —Bn, preferably —Boc.

Compound B-4 and Compound B-5 are subjected to a substitution reaction in a solvent (e.g., DMSO, NMP or DMF, and the like.) and under alkaline conditions to obtain Compound B-2. The alkali may be one or more selected from the group consisting of DIPEA, potassium carbonate, cesium carbonate, potassium phosphate and potassium tert-butanolate.

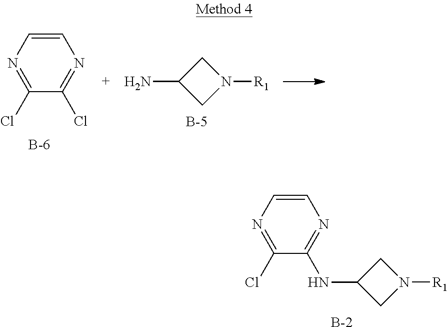

wherein, R₁ is a protecting group that can be one selected from the group consisting of —Boc, —Cbz, —Fmoc, trifluoroacetyl, —Trt, —PMB, and —Bn, preferably —Boc.

Compound B-6 and Compound B-5 are subjected to a substitution reaction under alkaline conditions to obtain Compound B-2.

The alkali may be one or more selected from the group consisting of DIPEA, TEA, potassium carbonate, cesium carbonate, potassium phosphate and potassium tert-butanolate, preferably DIPEA or TEA. The solvent for the substitution reaction may be one or more selected from the group consisting of DMSO, NMP and DMF.

In some embodiments, in the preparation method of the compound represented by Formula I, Compound A-1 may be prepared by the following steps:

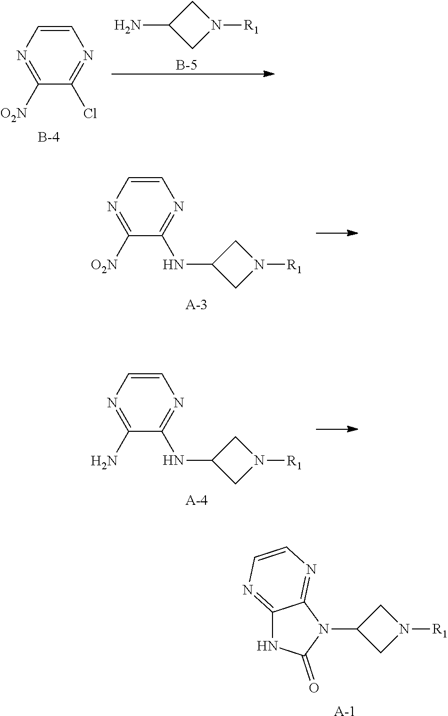

wherein, R₁ is a protecting group that can be one selected from the group consisting of —Boc, —Cbz, —Fmoc, trifluoroacetyl, —Trt, —PMB, and —Bn, preferably —Boc; and

Compound B-4 and Compound B-S are subjected to a substitution reaction in a solvent (e.g., DMSO, NMP or DMF, and the like) and under alkaline conditions to obtain Compound A-3, and then Compound A-3 is reduced by a reducing agent in a solvent (EtOH/H₂O or EtOH) to obtain Compound A-4, and Compound A-4 and CDI are subjected to a ring-closing reaction in a solvent (e.g., DMF or acetonitrile) to obtain Compound A-1.

The alkali may be one or more selected from the group consisting of DIPEA, potassium carbonate, cesium carbonate, potassium phosphate and potassium tert-butanolate, and the reducing agent may be selected from the group consisting of H₂—Pd/C, Fe and Zn.

The specific reaction conditions of the preparation route for the compound represented by Formula I are described in the specific embodiments. It should be understood that the reaction solvents and conditions applied in the preparation route are not limited to those applied in the specific embodiments, and other conventional reaction conditions of this type in the art may also be applicable to the preparation of the compound represented by Formula I.

Another aspect of the present application provides an intermediate that can be used in the preparation of the compound represented by Formula I, and the intermediate is selected from the group consisting of

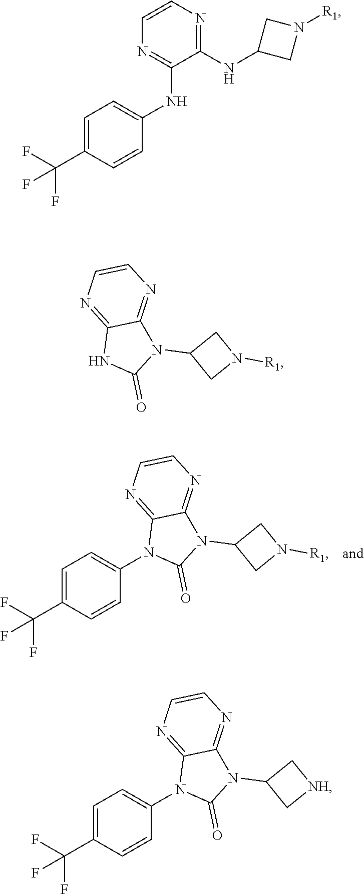

wherein, R₁ is a protecting group that can be one selected from the group consisting of —Boc, —Cbz, —Fmoc, trifluoroacetyl, —Trt, —PMB, and —Bn.

In some embodiments, R₁ is preferably —Boc.

Another aspect of the present application provides a preparation method of the crystal form A of the compound represented by Formula I.

In some embodiments, the compound represented by Formula I is slurried with methanol, filtered, and dried to obtain the crystal form A of the compound represented by Formula I.

In some embodiments, the compound represented by Formula I is dissolved by heating in a good solvent, then cooled down naturally, filtered after precipitating a solid, and dried to obtain the crystal form A.

In some embodiments, the compound represented by Formula I is dissolved by heating in a good solvent, then slowly added dropwise with a poor solvent, cooled down naturally, filtered after precipitating crystals, and dried to obtain the crystal form A.

The compound represented by Formula I as the raw material in the preparation method may be in any solid form obtained by any preparation method.

The good solvent is solvent A or solvent B, wherein,
solvent A is one or more selected from the group consisting of methanol, ethanol, acetonitrile, n-propanol, isopropanol, acetone, tetrahydrofuran, ethyl acetate, isopropyl acetate and butanone; and
solvent B is a mixture of solvent A and a poor solvent.

The poor solvent is one or more selected from the group consisting of H₂O, methyl tert-butyl ether, anisole, n-heptane and n-hexane,

### Use of the Pharmaceutical Composition, and Methods for Treating Solid Tumor Cancers, Hematologic Malignancies, Inflammatory Diseases, Autoimmune Diseases, and Other Diseases

The present application further provides use of the pharmaceutical composition in the manufacture of a medicament.

In some embodiments, the medicament can be used to treat a disease mediated by an abnormality of Hippo pathway.

In some embodiments, the medicament can be used to treat a disease mediated by YAP/TAZ and TEAD interaction, NF2 mutation/defect, LATS1/2 mutation/defect, LKBI mutation/defect, YAP/TAZ fusion, or abnormal activation of YAP/TAZ.

The present application further provides preferred technical solutions for the use. As a preferred embodiment, the medicament is for use in treating, preventing, delaying or stopping the onset or progression of cancer, cancer metastasis, proliferative diseases or inflammatory diseases.

As a preferred embodiment, the cancer is selected from the group consisting of colon cancer, stomach cancer, thyroid cancer, lung cancer, leukemia, pancreatic cancer, melanoma, multiple melanoma, brain cancer, kidney cancer, liver cancer, squamous cell carcinoma, gastrointestinal cancer, mesothelioma, skin cancer, prostate cancer, ovarian cancer, and breast cancer.

The present application also provides a method for treating and/or preventing a disease by administering a therapeutically effective amount of the pharmaceutical composition to a subject to be treated.

As a preferred embodiment, in the above method, the disease is a disease mediated by an abnormality of Hippo pathway.

As a preferred embodiment, in the above method, the disease is a disease mediated by YAP/TAZ and TEAD interaction, NF2 mutation/defect, LATS1/2 mutation/defect, LKBI mutation/defect, YAP/TAZ fusion, or abnormal activation of YAP/TAZ.

As a preferred embodiment, the disease is cancer, a proliferative disease or an inflammatory disease.

As a preferred embodiment, in the above method, the cancer is selected from the group consisting of colon cancer, stomach cancer, thyroid cancer, lung cancer, leukemia, pancreatic cancer, melanoma, multiple melanoma, brain cancer, kidney cancer, liver cancer, squamous cell carcinoma, gastrointestinal cancer, mesothelioma, skin cancer, prostate cancer, ovarian cancer, and breast cancer.

As a preferred embodiment, in the above method, the subject to be treated is human.

The term “disease” or “condition” or “symptom” as used herein refers to any illness, discomfort, disease, symptom or indication.

The term “therapeutically effective amount” as used herein refers to the amount of a compound that, when administered to a subject to be treated for treating a disease or at least one clinical symptom of a disease or condition, is sufficient to affect such treatment of the disease, condition or symptom. The “therapeutically effective amount” may vary with the compound, disease, condition and/or symptom of the disease or condition, the severity of the disease, condition and/or symptom of the disease or condition, the age of the patient being treated, and/or the weight of the patient being treated, and the like. In any particular case, a suitable amount may be apparent to those of skill in the art or may be determined using routine experiments. In the case of combination therapy, “therapeutically effective amount” means the total amount of effectively treating the disease, condition, or symptom of the subject in combination therapy.

The compound and/or crystal form described herein may be used alone or in combination as an active component to be mixed with a pharmaceutical carrier to form a pharmaceutical composition. Although the most suitable mode of administration of the active component in any given situation will depend on the particular subject being administered, the nature of the subject and the severity of the condition, the pharmaceutical composition of the present application includes a pharmaceutical composition suitable for oral, rectal, topical and parenteral (including subcutaneous, intramuscular, intravenous) administration. The pharmaceutical composition of the present application may conveniently be presented in unit dosage form well known in the art and prepared by any method of preparation well known in the art of pharmacy.

Generally, to treat the conditions or disorders indicated above, the dose level of the medicament is about 0.01 mg/kg body weight to 150 mg/kg body weight per day, or 0.5 mg to 7 g per patient per day. For example, for colon cancer, rectal cancer, skin cancer, mantle cell lymphoma, multiple myeloma, breast cancer, prostate cancer, glioblastoma, esophageal squamous cell carcinoma, liposarcoma, T-cell lymphoma melanoma, pancreatic cancer, malignant glioma, or lung cancer, the dose level of the medicament for effective treatment is 0.01 mg/kg body weight per day to 50 mg/kg body weight per day, or 0.5 mg/day per patient to 3.5 g/day per patient.

It will be appreciated, however, that lower or higher doses than those described above may be required. The specific dose level and treatment regimen for any particular patient will depend on a variety of factors, including the activity of the specific compound used, age, body weight, general health condition, gender, diet, time of administration, route of administration, excretion rate, medicament combination conditions and the severity of the specific disease being treated.

These and other aspects will become apparent from the following written description of the present application.

The following Examples are provided to better illustrate the present application. All parts and percentages are by weight and all temperatures are in degrees Celsius unless expressly stated otherwise.

The present application will be described in more detail by way of specific Examples. The following Examples are offered for illustrative purposes and are not intended to limit the present application in any manner. Those skilled in the art will readily recognize that various non-critical parameters can be varied or modified to produce substantially the same results. According to at least one determination method described herein, the compound in Examples is found to inhibit the transcription activity of protein-protein interaction of YAP/TAZ and TEAD.

### BRIEF DESCRIPTION OF THE DRAWINGS

FIG. 1: X-ray powder diffraction pattern of the crystal form A of the compound represented by Formula I.

FIG. 2: Thermogravimetric analysis (TGA) pattern of the crystal form A of the compound represented by Formula I.

FIG. 3: Differential scanning calorimetry (DSC) pattern of the crystal form A of the compound represented by Formula I.

FIG. 4: Projection pattern of cell stacking along the a direction for a single crystal of the crystal form A of the compound represented by Formula I.

FIG. 5: Ellipsoidal graph of the stereostructure of the asymmetric unit for a single crystal of the crystal form A of the compound represented by Formula I.

FIG. 6: X-ray powder diffraction pattern 1 of the crystal form A of the compound represented by Formula I prepared by different methods.

FIG. 7: X-ray powder diffraction pattern 2 of the crystal form A of the compound represented by Formula I prepared by different methods.

FIG. 8: DVS pattern of the crystal form A of the compound represented by Formula I.

FIG. 9: Inhibition curves of the compound represented by Formula I in NCI-H226 cell line.

FIG. 10: Tumor growth curves of Balb/c nude mice bearing NCI-H226 tumors in different treatment groups.

Unless otherwise indicated, information on the detection instruments and parameters of the detection methods used in the present application are as follows:

TABLE 1

| Equipment name | X-ray powder diffractometer (XRPD) | |
|---|---|---|
| Instrument | Bruker D8 Advance | |
| Technical indexes | Kα radiation (40 kV, 40 mA) of copper | |
| | target at a wavelength of 1.54 Å, θ-2θ | |
| | goniometer, Ni monochromator, Lynxeye | |
| | detector | |
| Calibration substance | Al₂O₃ | |
| Parameters | Detection angle | 3°-40° 2θ |
| | Step size | 0.02° 2θ |
| | Speed | 0.6 s.step⁻¹ or 0.3 s.step⁻¹ |
| | Divergent slit | 0.6 mm |
| | Soller slit | 2.5° |

TABLE 2

| | |
|---|---|
| Equipment name | Differential scanning calorimeter (DSC) |
| Instrument | Discovery DSC 2500 |
| Sample tray | Aluminum crucible |
| Protective gas | Nitrogen |
| Gas flow rate | 50 mL/min |
| Commonly used | Ramp 10° C. /min |
| detection methods | |

TABLE 3

| | |
|---|---|
| Equipment name | Thermogravimetric analyzer (TGA) |
| Instrument | Discovery TGA 550 |
| Sample tray | Platinum crucible + aluminum crucible |
| Protective gas | Nitrogen |
| Gas flow rate | 40 mL/min |
| Detection | Ramp 10° C./min |
| method | |

TABLE 4

| | |
|---|---|
| Equipment name | Dynamic vapor sorption analyzer (DVS) |
| Manufacturer | Surface Measurement Systems |
| Instrument | DVS Resolution |
| Sample tray | Aluminum crucible |
| Protective gas | Nitrogen |
| Gas flow rate | 200 sccm |

TABLE 5

| | |
|---|---|
| Instrument | Nuclear magnetic resonance spectrometer (NMR) |
| Model | Bruker Avance Neo 500 |
| Detection type | NMR Hydrogen Spectroscopy |
| Parameters | NS: 32 |
| | D1: 1 [sec] |
| | O1P: 6.175 ppm |
| | SW: 19.9955 ppm |
| | TE: 298 K |

TABLE 6

| | |
|---|---|
| Equipment name | High performance liquid chromatograph (HPLC) |
| Manufacturer | Waters |
| Model | H-Class |
| Instrument | Incubator with constant temperature and humidity |
| Model | Pentax KBF720 incubator with constant |
| | temperature and humidity |

### Single Crystal Detection

Single crystal data is collected at 296 K using a Bruker SMART APEX-II with Cu Ka radiation. The crystal structure is analyzed by the direct method (Shelxs97). The least squares method is used to correct the structural parameters and identify the atom types. The positions of all hydrogen atoms are obtained using geometric calculations.

### DETAILED DESCRIPTION

The present application is further described below by means of the given Examples, but the Examples do not constitute any limitation on the scope of the claimed protection of the present application. In specific Examples of the present application, the techniques or methods described are conventional techniques or methods and the like in the art, unless otherwise indicated. In the following Examples, the raw materials and reagents are obtained through commercially available purchases; the percentages, proportions, ratios or portions, and the like, are calculated by weight, unless otherwise stated. In the following Examples, the experimental temperature and humidity are room temperature and room humidity, unless otherwise stated.

### Abbreviations

BINAP: 1,1′-binaphthyl-2,2′-bisdiphenylphosphine;
BOP:
benzotriazol-1-yloxytris (dimethylamino) phosphonium hexafluorophosphate;
BrettPhos:
2-(dicyclohexylphosphino)-3,6-dimethoxy-2′,4′,6′-triisopropyl-1,1′-biphenyl;
Boc: tert-butoxycarbonyl;
Boc₂O: di-tert-butyl dicarbonate;
Bn: benzyl;
Cbz: benzyloxycarbonyl;
CDI: N,N-carbonyl diimidazole;
Cs₂CO₃: cesium carbonate;
DavePhos: 2-dicyclohexylphosphino-2′-(N,N-dimethylamino) biphenyl;
DCC: dicyclohexyl carbodiimide;
DCM: dichloromethane; DIPEA: N,N-diisopropylethylamine;
DMSO: dimethyl sulfoxide;
DME: 1,2-dimethoxyethane;
DMF: N,N-dimethylformamide;
DMK: acetone;
Dppf: 1,1′-bis(diphenylphosphino) ferrocene;
EA: ethyl acetate;
EDCI: 1-(3-dimethylaminopropyl)-3-ethylcarbodiimide hydrochloride;
EtOH: ethanol;
Fmoc: 9-fluorenylmethoxycarbonyl;
HATU:
2-(7-azobenzotriazole)-N,N,N′,N′-tetramethyluronium hexafluorophosphate;
HBTU: benzotriazol-N,N,N′,N′-tetramethylurea hexafluorophosphate;
HOBT: N-hydroxy-7-benzotriazole;
HOSu: N-hydroxysuccinimide;
HEX: n-hexane;
K₂CO₃: potassium carbonate;
K₃PO₄: potassium phosphate;
MeOH: methanol;
MEK: butanone;
MTBE: methyl tertiary butyl ether;
MsCl: methylsulfonyl chloride;
NMP: N-methylpyrrolidone;
NsCl: p-nitrobenzenesulfonyl chloride;
Pd₂(dba)₃: tris (dibenzylideneacetone) dipalladium;
Pd(OAc)₂: palladium acetate;
P(t-Bu)₃·HBF₄: tri-tert-butylphosphine tetrafluoroborate;
PyBOP: 1H-benzotriazol-1-yl oxytripyrrolidinyl hexafluorophosphate;
PMB: p-methoxybenzyl;
RuPhos: 2-dicyclohexylphospho-2′,6′-diisopropoxy-1,1′-biphenyl;
SPhos: 2-dicyclohexylphosphine-2′,6′-dimethoxy-biphenyl;
T₃P: 1-propylphosphonic anhydride;
TEA: triethylamine;
TFA: trifluoroacetic acid;
Trt: triphenylmethyl;
TsCl: p-toluenesulfonyl chloride;
2-MeTHF: 2-methyltetrahydrofuran;
t-BuOH: tert-butanol;
n-BuOH: n-butanol;
t-BuONa: sodium tert-butoxide;
XPhos: 2-dicyclohexylphosphino-2′,4′,6′-triisopropylbiphenyl;
Xantphos: 4,5-Bis (diphenylphosphino)-9,9-dimethylxanthene; RH: relative humidity;
RRT: relative retention time;
hrs/h: hours;
LC-MS/LCMS: liquid chromatography-mass spectrometry;
¹H-NMR/HNMR: nuclear magnetic resonance hydrogen spectroscopy;
XRD/XRPD: X-ray powder diffraction;
DSC: differential scanning calorimetry;
TGA: thermogravimetric analysis;
DVS: dynamic vapor sorption.

### Example 1 Preparation Method of Intermediate Compound 1-1

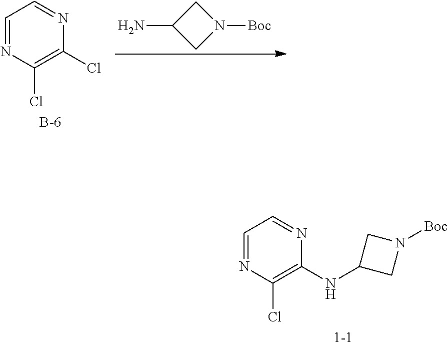

A mixture of 2,3-dichloropyrazine (1.05 g). 1-tert-butoxycarbonyl-3-amino-cyclobutanamine (1.46 g), DIPEA (2.46 mL) and DMSO (10 ml) was reacted at 110° C. for 16 h under nitrogen protection. After completion of the reaction, the reaction was cooled down naturally to room temperature and quenched by addition of water with stirring. A large amount of solid was precipitated and filtered. The filter cake was dried under vacuum at 50° C. to obtain 1.85 g of the target compound.

LC-MS [M+H⁺]: 285, LC-MS [M+H−56]⁺: 229.

¹H NMR (500 MHz, Chloroform-d) δ 7.95 (d, J=2.7 Hz, 1H), 7.66 (d, J=2.7 Hz, 1H), 5.48 (d, J=6.2 Hz, 1H), 4.67 (m, 1H), 4.41-4.29 (m, 2H), 3.83 (m, 2H), 1.46 (s, 9H).

### Example 2 Preparation Method of Intermediate Compound 1-1 and Intermediate Compound 2-1

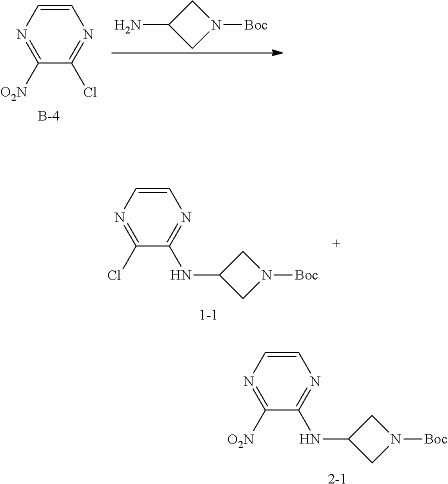

A mixture of 2-chloro-3-nitropyrazine (10.00 g), tert-butyl 3-aminoazetidine-1-carbonate (11.88 g), DMF (200 mL) and potassium carbonate (17.33 g) was stirred at room temperature overnight. After completion of the reaction, the reaction solution was poured into water, and the resultant was extracted with ethyl acetate. The organic phases were combined. The combined organic phase was washed with water, dried with anhydrous sodium sulfate, and filtered. The filtrate was concentrated under reduced pressure and the resulting crude product was purified and separated by column chromatography (EA/HEX=0% to 40%) to obtain 4.12 g of Compound 1-1 (LC-MS [M+H-56]⁺: 229) and 13.32 g of Compound 2-1 (LC-MS [M+H−56]⁺: 240).

### Example 3 Synthesis of the Compound Represented by Formula I

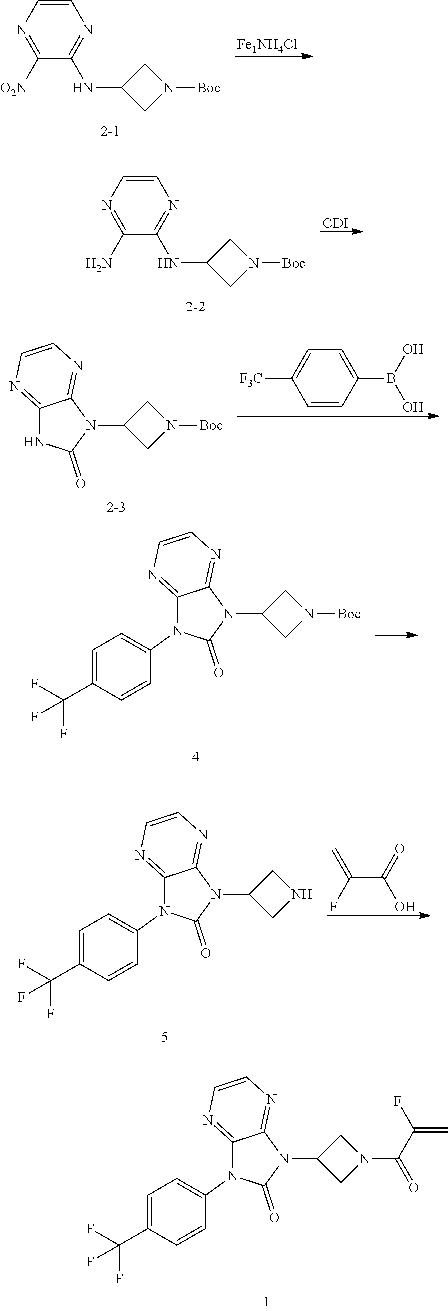

### Step 1: Preparation of Compound 2-2

A mixture of Compound 2-1 (1.33 g), iron powder (1.45 g), NH₄Cl (1.39 g), EtOH (20 mL) and water (5 mL) was heated to 80° C. and reacted for 3 h. After completion of the reaction, the reaction solution was cooled down to room temperature and filtered with diatomite. The filtrate was concentrated under reduced pressure and the resulting crude product was purified by column chromatography (EA/HEX=0% to 40%) to obtain the target Compound 2-2 (0.69 g).

LCMS [M+H−56]⁺: 210.

### Step 2: Preparation of Compound 2-3

Compound 2-2 (0.69 g) was dissolved in DMF (60 mL) and heated to 75° C. with stirring. CDI (1.26 g) was added to the solution and the reaction was carried out for 3 h. After completion of the reaction, the reaction solution was poured into ice water, and the resultant was extracted with ethyl acetate. The organic phases were combined, and the combined organic phase was washed with water, dried with anhydrous sodium sulfate, and filtered. The filtrate was concentrated under reduced pressure and the resulting crude product was purified by column chromatography (EA/HEX=0% to 50%) to obtain the target Compound 2-3 (0.60 g).

LCMS [M+H−56]⁺: 236.

### Step 3: Preparation of Compound 4

A mixture of Compound 2-3(300 mg), 4-trifluoromethylphenylboronic acid (391 mg), Cu(OAc)₂ (187 mg), triethylamine (0.43 mL) and dichloromethane (10 mL) was reacted with stirring at room temperature for 14 h under oxygen atmosphere. After completion of the reaction, the reaction mixture was concentrated under reduced pressure and the resulting crude product was purified by column chromatography (EA/HEX=0% to 30%) to obtain the target Compound 4(411 mg).

LCMS [M+H−56]⁺: 380.

### Step 4: Preparation of Compound 5

A mixture of Compound 4 (0.41 g), dichloromethane (4 mL), and trifluoroacetic acid (0.75 mL) was reacted overnight with stirring at room temperature. After completion of the reaction, the reaction solution was concentrated under reduced pressure. The concentrated residue was dissolved by adding dichloromethane and the pH was adjusted to 10 to 12 by adding saturated aqueous potassium carbonate. The solution was allowed to stand for phase separation, and the organic phase was separated. The aqueous phase was extracted with dichloromethane. The organic phases were combined, and the combined organic phase was washed with water, dried with anhydrous sodium sulfate, and filtered. The filtrate was concentrated under reduced pressure and the resulting crude product was purified by column chromatography (DCM/MeOH=0% to 10%) to obtain the target Compound 5 (0.25 g).

LCMS [M+H]⁺: 336.

¹H NMR (500 MHz, Chloroform-d) δ 8.09 (d. J=3.2 Hz, 1H), 8.02 (d, J=3.2 Hz, 1H), 7.97 (d, J=8.4 Hz, 2H), 7.80 (d, J=8.5 Hz, 2H), 5.52 (p, J=7.9 Hz, 1H), 4,75-4.62 (m, 2H), 3.93 (t,)=8.2 Hz, 2H), 2.26 (6, 1H).

### Step 5: Preparation of the Compound Represented by Formula I

A mixture of 2-fluoroacrylic acid (0.24 g), DIPEA (0.69 g), DMF (5 mL), HATU (0.81 g) and Compound 5 (0.36 g) was reacted with stirring for 0.5 h at room temperature. After completion of the reaction, the reaction was quenched with water, and the resultant was extracted with ethyl acetate. The organic phases were combined, and the combined organic phase was washed with water, dried with anhydrous sodium sulfate, and filtered. The filtrate was concentrated under reduced pressure and then purified by column chromatography (EA/HEX=0% to 50%) to obtain 0.25 g of the target compound represented by Formula I.

¹H NMR (500 MHZ, DMSO) δ 8.16-8.11 (m, 1H), 8.05 (d, J=3.2 Hz, 1H), 7.99 (q, J=8.7 Hz, 4H), 5.54 (dd, J=48.5, 3.5 Hz, 1H), 5.45 (dq, J=8.7, 5.8 Hz, 1H), 5.35 (dd, J=16.6, 3.5 Hz, 1H), 5.05-4.97 (m, 1H), 4.79 (td, J=9.3, 4.2 Hz, 1H), 4.67 (dd, J=10.6, 5.8 Hz, 1H), 4.43 ((J=9.7 Hz, 1H).

LC-MS [M+H]⁺: 408.

### Example 4 Synthesis of the Compound Represented by Formula I

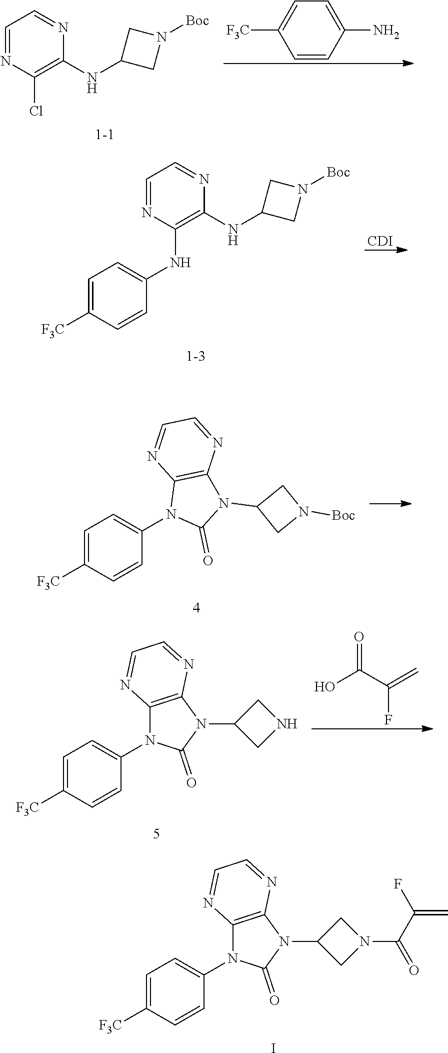

### Step 1: Preparation of Compound 1-3

A mixture of Compound 1-1(1.70 g), 4-trifluoromethylaniline (1.15 g), potassium phosphate (3.80 g), tris (dibenzylideneacetone) dipalladium (0.28 g), Xantphos (0.35 g) and toluene (10 mL) was reacted at 100° C. for 5 h under nitrogen protection. After completion of the reaction, the reaction was quenched by adding water, and the resultant was extracted with ethyl acetate. The organic phase was washed with water, dried with anhydrous sodium sulfate, and filtered. The filtrate was concentrated under reduced pressure and the resulting crude product was purified by column chromatography (HEX:EA=0% to 30%) to obtain the target Compound 1-3 (1.90 g).

LC-MS [M+H]⁺: 410.

¹H NMR (500 MHZ, Chloroform-d) 6 7.65 (d, J=8.6 Hz, 2H), 7.61-7.57 (m, 2H), 7.56-7.49 (m, 3H), 5.79 (d, J=6.1 Hz, 1H), 4.65 (ddd, J=10.0, 5.4, 2.9 Hz, 1H), 4.39 (s, 2H), 3.82 (s, 2H), 1.47 (s, 9H).

### Step 2: Preparation of Compound 4

A mixture of Compound 1-3 (0.82 g), triethylamine (1.01 g), acetonitrile (10 mL) and CDI (0.98 g) was heated to reflux and reacted for 4 h. After completion of the reaction, the reaction solution was cooled down to room temperature. The reaction was quenched by adding water, and the resultant was extracted with ethyl acetate. The organic phase was washed with water, dried with anhydrous sodium sulfate, and filtered. The filtrate was concentrated under reduced pressure and the resulting crude product was purified by column chromatography (EA/HEX=0% to 30%) to obtain the target Compound 4 (0.78 g).

LC-MS [M+H−56]⁺: 380.

¹H NMR (500 MHZ, Chloroform-d) δ 8.08 (d, J=3.2 Hz, 1H), 8.03 (d, J=3.2 Hz, 1H), 7.99-7.94 (m, 2H), 7.84-7.78 (m, 2H), 5.39 (m, III), 4.77 (m, 2H), 4.36 (m, 2H), 1.50 (m, 9H).

### Step 3: Preparation of Compound 5

A mixture of Compound 4(0.65 g), dichloromethane (5 mL), and trifluoroacetic acid (1 mL) was reacted overnight with stirring at room temperature. After completion of the reaction, the reaction solution was concentrated under reduced pressure. The concentrated residue was dissolved by adding dichloromethane and the pH was adjusted to 10 to 12 by adding saturated aqueous potassium carbonate. The solution was allowed to stand for phase separation. The organic phase was separated, and the water phase was extracted with dichloromethane. The organic phases were combined, and the combined organic phase was washed with water, dried with anhydrous sodium sulfate, and filtered. The filtrate was concentrated under reduced pressure and the resulting crude product was purified by column chromatography (DCM/MeOH=0% to 10%) to obtain the target Compound 5 (0.36 g).

LC-MS [M+H]⁺: 336.

¹H NMR (500 MHZ, Chloroform-d) δ 8.09 (d. J=3.2 Hz, 1H), 8.02 (d, J=3.2 Hz, 1H), 7.97 (d, J=8.4 Hz, 2H), 7.80 (d, J=8.5 Hz, 2H), 5.52 (p, J=7.9 Hz, 1H), 4.75-4.62 (m, 2H), 3.93 (t,/=8.2 Hz, 2H), 2.26 (s, 1H).

### Step 4: Preparation of the Compound Represented by Formula I

A mixture of 2-fluoroacrylic acid (0.24 g), DIPEA (0.69 g), DMF (5 mL), HATU (0.81 g) and Compound 5 (0.36 g) was reacted with stirring for 0.5 h at room temperature. After completion of the reaction, the reaction was quenched by adding water, and the resultant was extracted with ethyl acetate. The organic phases were combined, and the combined organic phase was washed with water, dried with anhydrous sodium sulfate, and filtered. The filtrate was concentrated under reduced pressure to obtain the crude product, and the obtained crude product was purified and separated by high-pressure preparative liquid phase (mobile phase A: 0.1% aqueous formic acid solution; mobile phase B: acetonitrile, detection wavelength of 254 nM, gradient: 35% to 65%, flow rate: 40 mL/min, packed column: luna C18(3), 30×250) to obtain the target compound represented by Formula I (0.28 g).

### LC-MS [M+H]⁺: 408.

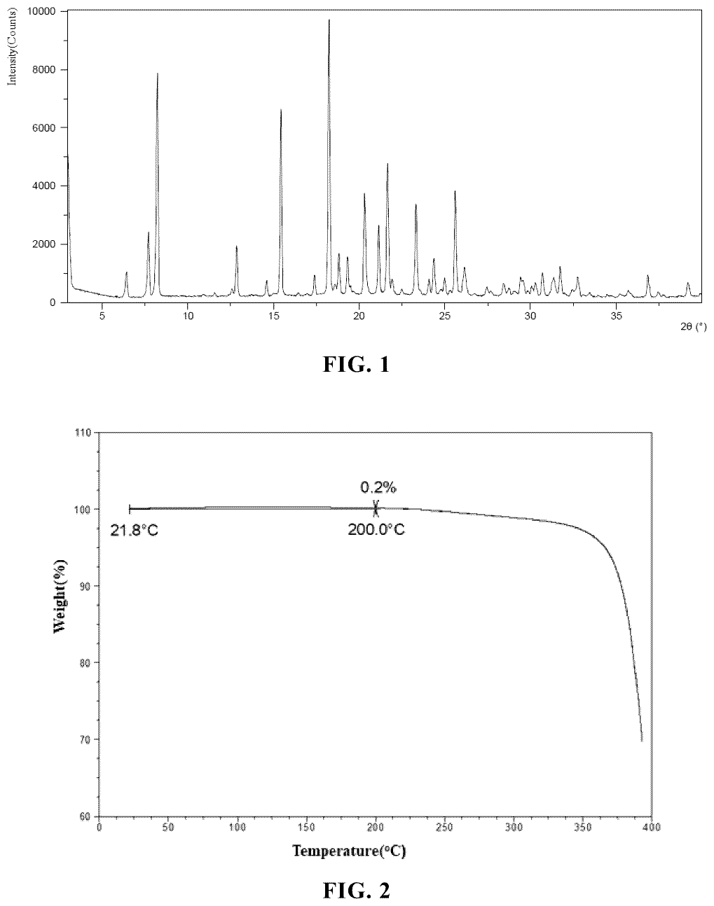

The target compound represented by Formula I obtained in Example 4 was characterized by XRPD and has an XRPD pattern substantially as shown in FIG. 1, and the peak temperature of DSC melting endotherm is 170.7° C. It is shown that the target compound obtained is the crystal form A of the compound represented by Formula I.

The primary data of X-ray powder diffraction pattern of the crystal form A of the compound represented by Formula 1 is shown in Table 7.

TABLE 7

| Number | 2θ ± 0.2 (°) | Inter Planar Spacing [Å] | Relative intensity (%) |
|---|---|---|---|
| 1 | 6.413 | 13.77205 | 8.2 |
| 2 | 7.697 | 11.47658 | 21.7 |
| 3 | 8.218 | 10.74973 | 74.7 |
| 4 | 10.881 | 8.12471 | 0.6 |
| 5 | 11.547 | 7.65759 | 1.2 |
| 6 | 12.569 | 7.03682 | 2.5 |
| 7 | 12.842 | 6.88803 | 17.6 |
| 8 | 14.591 | 6.06618 | 5.2 |
| 9 | 15.427 | 5.73909 | 64.7 |
| 10 | 16.435 | 5.38945 | 0.9 |
| 11 | 17.383 | 5.09748 | 7.0 |
| 12 | 18.231 | 4.86235 | 100.0 |
| 13 | 18.581 | 4.77140 | 3.4 |
| 14 | 18.812 | 4.71342 | 14.7 |
| 15 | 19.310 | 4.59295 | 13.5 |
| 16 | 20.306 | 4.36974 | 36.4 |
| 17 | 21.131 | 4.20101 | 25.2 |
| 18 | 21.641 | 4.10321 | 48.4 |
| 19 | 21.915 | 4.05252 | 5.1 |
| 20 | 22.471 | 3.95349 | 1.8 |
| 21 | 23.306 | 3.81372 | 34.4 |
| 22 | 24.059 | 3.69605 | 6.1 |
| 23 | 24.345 | 3.65316 | 14.3 |
| 24 | 24.977 | 3.56219 | 6.6 |
| 25 | 25.590 | 3.47819 | 45.3 |
| 26 | 26.136 | 3.40677 | 10.0 |
| 27 | 27.440 | 3.24774 | 3.4 |
| 28 | 28.420 | 3.13801 | 4.1 |
| 29 | 29.410 | 3.03453 | 7.7 |
| 30 | 30.682 | 2.91160 | 9.2 |

### Example 5 Preparation Method of the Crystal Form a of the Compound Represented by Formula I

### Preparation Method 1

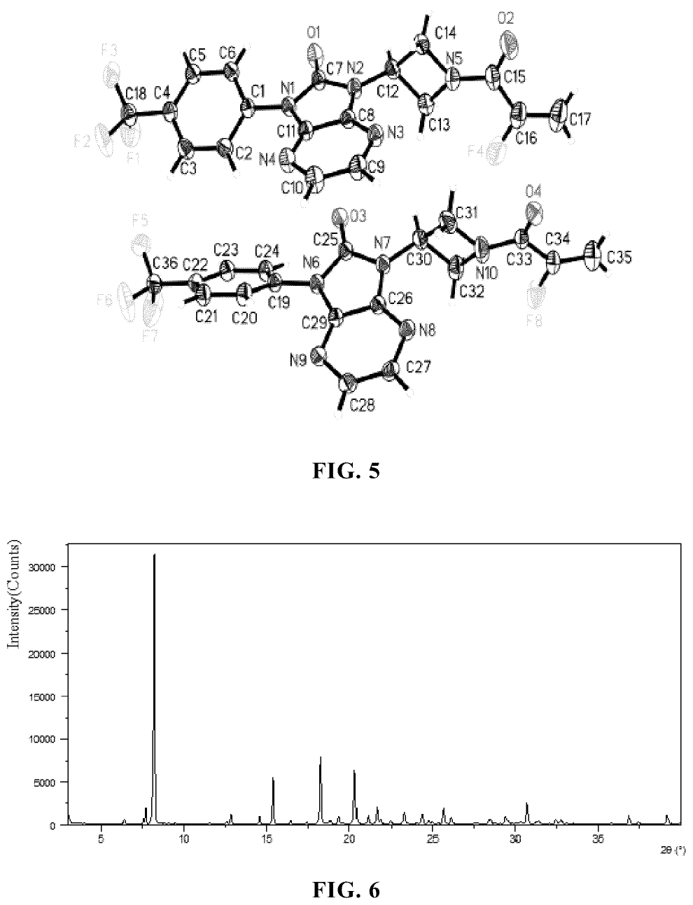

The crude product obtained by concentration under reduced pressure in step 4 of Example 4 was added to methanol for slurrying, and the resultant was filtrated and dried under vacuum to obtain the crystal form A of the compound represented by Formula 1. After XRPD detection, an XRPD pattern substantially as shown in FIG. 6 was obtained, the primary data was as shown in Table 8, and the peak temperature of DSC melting endotherm was 170.7° C.

TABLE 8

| Number | 2θ ± 0.2 (°) | Inter Planar Spacing [Å] | Relative intensity (%) |
|---|---|---|---|
| 1 | 6.396 | 13.80828 | 1.7 |
| 2 | 7.672 | 11.51405 | 5.6 |
| 3 | 8.192 | 10.78415 | 100.0 |
| 4 | 11.533 | 7.66644 | 0.3 |
| 5 | 12.576 | 7.03302 | 1.0 |
| 6 | 12.837 | 6.89087 | 3.9 |
| 7 | 14.565 | 6.07681 | 2.9 |
| 8 | 15.394 | 5.75115 | 18.1 |
| 9 | 16.448 | 5.38491 | 1.3 |
| 10 | 17.393 | 5.09459 | 0.8 |
| 11 | 18.249 | 4.85753 | 25.8 |
| 12 | 18.576 | 4.77271 | 0.5 |
| 13 | 18.813 | 4.71316 | 1.5 |
| 14 | 19.324 | 4.58970 | 2.7 |
| 15 | 20.317 | 4.36758 | 21.1 |
| 16 | 21.139 | 4.19950 | 3.4 |
| 17 | 21.681 | 4.09564 | 6.3 |
| 18 | 21.880 | 4.05887 | 2.1 |
| 19 | 22.451 | 3.95686 | 1.4 |
| 20 | 23.310 | 3.81298 | 5.2 |
| 21 | 24.045 | 3.69810 | 0.6 |
| 22 | 24.381 | 3.64793 | 4.1 |
| 23 | 24.987 | 3.56077 | 1.0 |
| 24 | 25.648 | 3.47047 | 7.0 |
| 25 | 26.111 | 3.40995 | 2.8 |
| 26 | 27.507 | 3.24004 | 0.3 |
| 27 | 28.458 | 3.13382 | 2.2 |
| 28 | 29.391 | 3.03646 | 3.5 |
| 29 | 30.682 | 2.91156 | 9.1 |

### Preparation Method 2

The compound represented by Formula 1 (2.62 g) prepared by the method of Example 3 was added to EA (15 mL), and the resultant was heated to 80° C., cooled down naturally to precipitate crystal, filtrated and dried to obtain the crystal form A of the compound represented by Formula 1. After XRPD detection, an XRPD pattern substantially as shown in FIG. 7 was obtained, the primary data was as shown in Table 9, and the peak temperature of the DSC melting endotherm was 170.8° C.

TABLE 9

| Number | 2θ ± 0.2 (°) | Inter Planar Spacing [Å] | Relative intensity (%) |
|---|---|---|---|
| 1 | 6.356 | 13.89575 | 13.6 |
| 2 | 7.631 | 11.57519 | 6.4 |
| 3 | 8.145 | 10.84655 | 30.4 |
| 4 | 10.882 | 8.12347 | 0.1 |
| 5 | 11.503 | 7.68653 | 0.8 |
| 6 | 12.536 | 7.05525 | 1.7 |
| 7 | 12.796 | 6.91239 | 30.7 |
| 8 | 14.528 | 6.09208 | 1.1 |
| 9 | 15.361 | 5.76356 | 19.8 |
| 10 | 16.422 | 5.39365 | 0.7 |
| 11 | 17.366 | 5.10251 | 0.6 |
| 12 | 18.217 | 4.86583 | 100.0 |
| 13 | 18.548 | 4.77977 | 1.2 |
| 14 | 18.784 | 4.72020 | 1.1 |
| 15 | 19.286 | 4.59852 | 24.0 |
| 16 | 20.288 | 4.37362 | 18.3 |
| 17 | 21.106 | 4.20587 | 2.2 |
| 18 | 21.653 | 4.10084 | 6.3 |
| 19 | 21.847 | 4.06500 | 1.5 |
| 20 | 22.446 | 3.95779 | 0.3 |
| 21 | 23.276 | 3.81846 | 2.5 |
| 22 | 24.012 | 3.70318 | 0.5 |
| 23 | 24.350 | 3.65242 | 17.1 |
| 24 | 24.958 | 3.56491 | 1.2 |
| 25 | 25.613 | 3.47513 | 7.3 |
| 26 | 26.084 | 3.41341 | 4.8 |
| 27 | 27.451 | 3.24648 | 0.6 |
| 28 | 28.423 | 3.13765 | 2.1 |
| 29 | 29.370 | 3.03859 | 1.4 |
| 30 | 30.665 | 2.91319 | 2.8 |

Notably, the crystal form A of the compound represented by Formula I of the present application has a stronger preferred orientation, it can be seen from FIGS. 1, 6, and 7 that the intensities of several peaks varied considerably in the XRPD pattern of the different samples but did not affect the matching of the entire pattern. In the present application, the solid form of the compound was verified by thermal analysis experiment and capillary XRPD experiment after elimination of the preferred orientation, both of which indicated the same crystal form A.

Therefore, the intensity data for the characteristic peaks in FIGS. 1, 6, and 7 are all exemplary and cannot be used as absolute comparisons. When the samples and/or detection conditions are different, due to the effect of preferred orientation, the intensities of the peaks reflected in the XRPD pattern may show some changes, e.g., some of the non-strong peaks may show higher intensities compared to those in FIG. 1, and/or some of the strong peaks may become weaker, but this does not affect the matching of the whole pattern.

### Example 6 Hygroscopicity Test of the Crystal Form A of the Compound Represented by Formula I

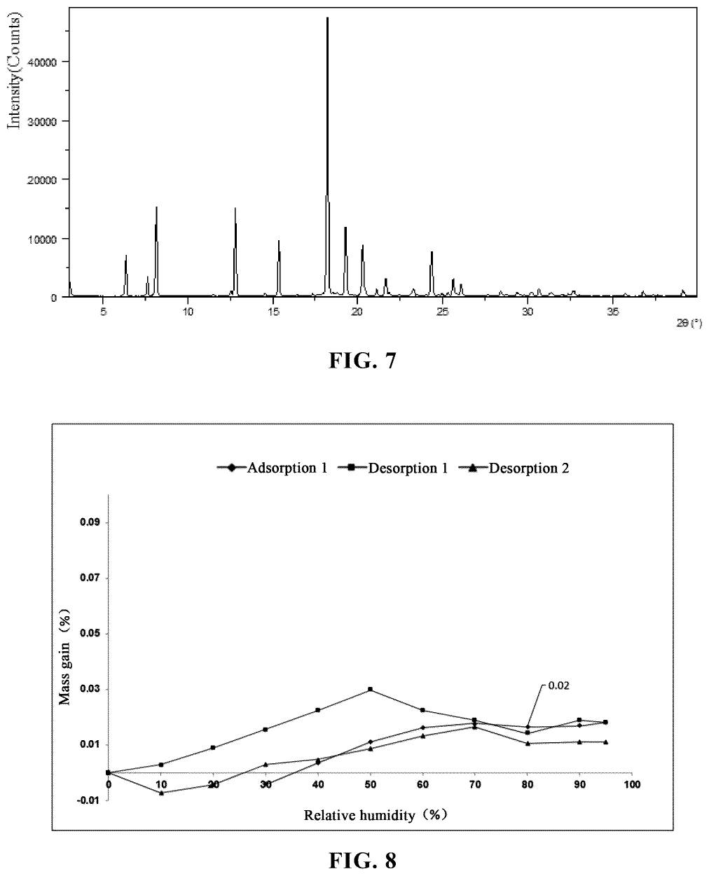

The DVS test on the crystal form A was performed using the dynamic vapor sorption analyzer and experimental conditions exemplified in Table 4, and a DVS plot substantially as shown in FIG. 8 was obtained, which showed that the crystal form A has no hygroscopicity. After the DVS test, no change in the crystal form was seen by XRPD detection.

### Example 7 Physicochemical Stability Test of the Crystal Form A of the Compound Represented by Formula I

Referring to the requirements of “9001 Guidelines for Stability Test of APIs and Pharmaceutical Preparations” in the Chinese Pharmacopoeia, the stability test was conducted on the crystal form A of the compound represented by Formula I, and the specific test conditions are shown in Table 10.

TABLE 10
Conditions and methods of stability test on the crystal
form A of the compound represented by Formula I

| | | Placement | Investigation | Investigation |
|---|---|---|---|---|
| Item | | conditions | duration | items |
| Influencing | High | Temperature: | Measurement | Characteristics, |
| factors | temperature | 40° C. | on day 5, day | weight gain/loss, |
| experiment | High | Temperature: | 10 and day | moisture, related |
| | temperature | 60° C. | 30, | substances, |
| | High | Humidity: 92.5% | respectively. | content, crystal |
| | humidity | RH | | form, and the |
| | High | Humidity: 75% | | like |
| | humidity | RH | | |
| | Illumination | Illumination of | | |
| | | 4500Lx | | |

| Accelerated test | 40° C. ± 2° C., | Months 1, 2, | Characteristics, |
|---|---|---|---|
| | RH75% ± 5% | 3, and 6 | moisture, related |
| Long-term test | 25° C. ± 2° C., | Months 3 and | substances, |
| | RH75% ± 5% | 6 | content, crystal |
| | | | form, and the |
| | | | like |

The crystal form A of the compound represented by Formula I was placed under conditions of the influencing factors, accelerated test, and long-term test, and there was no significant change in the crystal form, characteristics, and content compared with those on day 0, indicating that the crystal form A has excellent physicochemical stability.

### Example 8 Pressure Stability of the Crystal Form A of the Compound Represented by Formula I

The crystal form A of the compound represented by Formula I was placed under pressure conditions of 150 kg and 230 kg for pressing, and it was found by testing that the crystal form was not changed, the experimental results showed that the crystal form A of the compound represented by Formula I has excellent pressure stability.

### Example 9 Pharmacological Test of the Compound Represented by Formula I

### Example A: CTGF Detection (ELISA Method)

The detection of CTGF expression level may assess the activity of the YAP/TAZ-TEAD transcription complex. CTGF expression level was quantitatively detected using a human SimpleStep ELISA® kit (Abcam, ab261851).

NCI-H2052 cells (purchased from ATCC, NF2 mutation) were cultured in RPMI 1640 complete medium (containing 10% FBS, 1% penicillin-streptomycin solution and I mM sodium pyruvate). The day before compound treatment, cultured cells were washed with PBS, digested with trypsin, and then collected by centrifugation. The supernatant was removed, and the cells were resuspended in fresh complete medium. Cells were counted and inoculated in 96-well plates at a density of 6500 cells/well. The cells were then cultured overnight in an incubator (37° C., 5% CO₂).

After the cells were cultured overnight, the culture supernatant was discarded, the cells were washed with PBS solution, and 200 μL of medium containing the compound represented by Formula I was added to each well to culture the cells. The starting concentration of the compound represented by Formula I was 4 μM, and 3-fold cascade dilution was performed to obtain a total of 8 concentration gradients. The cells were then incubated in an incubator (37° C., 5% CO₂). After 24 hours of incubation, the cells were centrifuged at 4° C. and 1500 RPM for 5 min, and then 50 μL of culture supernatant was taken for CTGF ELISA detection.

The Human CTGF ELISA Kit uses an affinity labeled capture antibody and a reporter gene coupled detection antibody to immunocapture the sample analytes in the solution. To perform the detection, firstly, all reagents, samples and controls were prepared according to the kit instructions. 50 μL of standard or cell supernatant sample to be detected were added to the wells of the detection plate. 50 AL of antibody Cocktail was then added to each well. The plate was sealed and incubated on a plate shaker for 1 h at room temperature. At the end of the incubation, each well was washed 3 times with washing buffer. 100 μL of TMB developing solution was added to each well and the incubation was carried out on a plate shaker for 10 minutes away from light. 100 μL of termination solution was then added to each well. Shaking was performed on a plate shaker for 1 minute for mixing. Finally, the OD value at 450 nm of each well was read on a multifunctional microplate reader. The concentrations of the target CTGF protein in the samples were determined by standard curve.

The inhibitory activity of the compound of the present application on the transcription regulatory function of TEAD-YAP/TAZ was assessed by the CTGF concentration response curve of the compound. EC₅₀ value was calculated by fitting the concentration response curves using software GraphPad Prism. The EC₅₀ value of the compound represented by Formula I of the present application was detected as 2 nM, showing excellent inhibitory activity.

### Example B Cell Proliferation Inhibitory Activity Detection (CTG Detection)

The inhibition of the compound on cell proliferation was detected using CellTiter-Glo® Luminescent Cell Viability Assay Kit (Promega Company). NCI-H226 cells (purchased from ATCC, NF2 expression-deficient) were cultured in RPMI 1640 complete medium (containing 10% FBS, 1% penicillin-streptomycin solution and 1 mM sodium pyruvate).
a) The day before compound treatment, cultured cells were washed with PBS, digested with trypsin, and then collected by centrifugation. The supernatant was removed and the cells were resuspended in fresh RPMI 1640 complete medium. After cell counting, NCI-H226 cells were inoculated in 96-well plates at a density of 2000/well.
b) After 24 hours, the compound represented by Formula I diluted with complete medium to different concentrations was added to each well (the compound was first dissolved with DMSO to formulate a mother liquor, and then diluted with medium to a final concentration of 0.5% DMSO). The starting concentration of the compound represented by Formula I was 20 AM and 3-fold cascade dilution was performed to obtain a total of 10 concentrations.
c) The cells were cultured in an incubator (37° C., 5% CO₂) for 144 h.
d) After 144h, CTG detection was performed: first, CellTiter Glo® Buffer was added to CellTiter Glo® Substrate to prepare the detection reagent according to CellTiter-Glo® instructions. 50 μL of detection reagent was then added into each well of the 96-well plate, shaken gently for 2 min, and then placed at room temperature for 10 min. Finally, the luminescence signal value (RLU) of each well was read on a multifunctional microplate reader PE En Vision®.

### Data Analysis:

Inhibition rate (IR) of the detected compound: IR (%)=(1−(RLU compound−RLU blank control)/(RLU solvent control−RLU blank control))*100%. The inhibition rates of the compounds at different concentrations were calculated in Excel, then software GraphPad Prism was used to generate inhibition curves, and the relevant parameters were calculated, including the minimum inhibition rate Bottom. the maximum inhibition rate Top and IC₅₀.

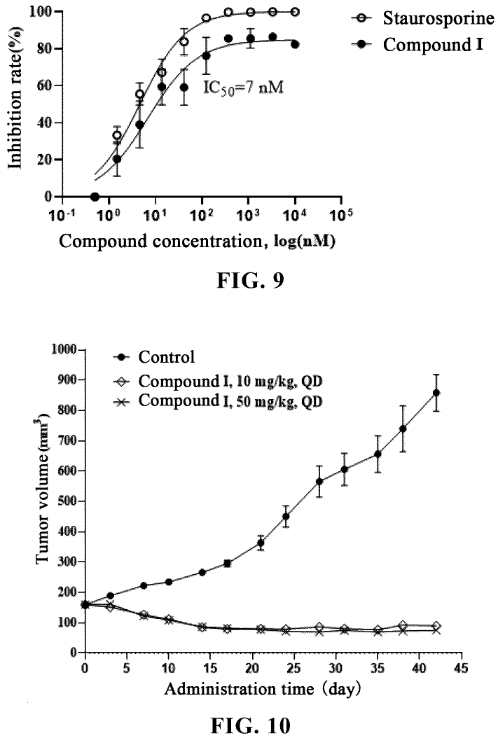

After detection, the inhibition curves of the compounds represented by Formula I in NCI-H226 cells was shown in FIG. 9, where the X-axis is compound concentration (nM), the Y-axis is inhibition rate %, and its IC₅₀ value was 7 nM, showing excellent inhibitory activity.

### Example C Pharmacokinetics Study of the Compound in Plasma of Mice after Oral Administration

Adult BALB/c female mice (6 weeks to 7 weeks) were received a single administration of the compound represented by Formula I containing 10% to 20% DMSO, 10% Solutol (KollipHor HS15) and 80% to 70% physiological saline as excipient. Mice (n=3) were administered orally (gavage, clear solution) at a dose of 5 mg/kg. Blood was collected after oral administration at the following points of time: 15 min, 30 min, 1 h, 2 h, 4 b, 7 h, and 24 h. About 0.1 mL of whole blood was collected from the retro-orbital venous plexus and placed in a test tube containing EDTA anticoagulant. Samples were centrifuged at 4° C. and 4000 rpm for 10 min. Plasma was transferred to a centrifugal tube and stored at −20° C. before analysis. The concentration of test compound in plasma samples was analyzed by liquid chromatography-tandem mass spectrometry (LC-MS/MS). Plasma concentration-time data from individual animals were analyzed using software Sciex Analyst. A non-compartmental model was introduced in the concentration analysis, and pharmacokinetic parameters of the test compound were calculated using software WinNonlin (version 4.1; pHarsight). The compound represented by Formula I had a Cmax of 1967 ng/ml and an AUClast of 17195 h*ng/ml, showing good pharmacokinetic characteristic.

### Example D In Vivo Pharmacodynamics and Efficacy Studies

In vivo pharmacodynamics and efficacy studies of the compound represented by Formula I were performed in a subcutaneous NCI-H226 (NF2 expression-deficient) human lung squamous cell carcinoma xenograft model in BALB/c nude mice.

### Method

BALB/c nude female mice (6 weeks to 7 weeks) were inoculated subcutaneously with 1×10⁷ NCI-H226 tumor cells on the right side of each. The inoculated cells were resuspended in a mixture solution of 0.1 mL of PBS and matrix gel Matrigel (PBS: Matrigel=1:1). Administration treatment was started in groups when the average tumor size reached about 100 mm³ to 200 mm³. From the date of grouping, the mice were orally administered the crystal form A of the compound represented by Formula I (solvent: 10% DMSO+10% Solutol (KollipHor HS15)+80% physiological saline) once a day for a total of 45 days (QD×45 days). Animals were regularly monitored for changes in body weight as an indicator of compound safety. Tumors were measured twice weekly using a caliper, and the volume was expressed in mm³ using the formula “V=(L×W\hat{ }2)/2”, where V is the tumor volume, L is the length of the tumor (the longest tumor dimension) and W is the width of the tumor (the longest tumor dimension perpendicular to L). Compound inhibitory activity was assessed by tumor growth inhibition TGI (%). TGI (%)=[1−(Ti−T0)/(Vi−V0)]×100; Ti is the mean tumor volume of a dosing group on a given day, TO is the mean tumor volume of the dosing group on day 0, Vi is the mean tumor volume of the control group on the same day as Ti, and V0 is the mean tumor volume of the control group on day 0. The results are shown in Table 11 and FIG. 10.

TABLE 11

| Administration | | |
|---|---|---|
| dosage (mg/kg) | TGI (%) | p value |
| 10 mg/kg, QD | 109% | <0.0001 |
| 50 mg/kg, QD | 111% | <0.0001 |

### Result

On day 45 after treatment, significant antitumor activity was observed in the groups of 10 mg/kg and 50 mg/kg based on the tumor volumes compared to the control group. The p-values were <0.0001. The TGI values were 109% and 111%, respectively.

### Example 10: Composition of the Crystal Form a of the Compound Represented by Formula I

Tablet formulation is shown in Table 12 below:

TABLE 12

| Component | Amount (g) |
|---|---|
| Batch (number | 20000 |
| of tablet) | |
| API ¹ | 400 |
| Lactose | 7120 |
| Cross-linked | 400 |
| polyvinyl ketone | |
| Magnesium | 80 |
| stearate | |
| Total weight | 8000 |

¹ The API described herein is specifically referred to the crystal form A of the compound represented by Formula I.

The tablet was prepared as follows: a prescription quantity of API, lactose and cross-linked polyvinyl ketone were mixed, sieved and further mixed. Then sieved magnesium stearate was added, the lubricated mixture was then pressed to prepare the tablet according to the theoretical tablet weight.

### Example 11: Composition of the Crystal Form a of the Compound Represented by Formula I

Tablet formulation is shown in Table 13 below:

TABLE 13

| Component | Amount (g) |
|---|---|
| Batch (number | 20000 |
| of tablet) | |
| API ¹ | 3600 |
| Mannitol | 1440 |
| Microcrystalline | 600 |
| cellulose | |
| Cross-linked | 300 |
| polyvinyl ketone | |
| Magnesium | 60 |
| stearate | |
| Total weight | 6000 |

¹ The API described herein is specifically referred to the crystal form A of the compound represented by Formula I.

The tablet was prepared as follows: a prescription quantity of API, mannitol, microcrystalline cellulose, and cross-linked polyvinyl ketone were mixed, sieved and further mixed. Then sieved magnesium stearate was added, the lubricated mixture was then pressed to prepare the tablet according to the theoretical tablet weight.

### Example 12: Stability Test of the Composition

Referring to the requirements of “9001 Guidelines for Stability Test of APIs and Pharmaceutical Preparations” in the Chinese Pharmacopoeia, the stability test was conducted for the composition of the crystal form A of the compound represented by Formula I, the experimental results showed that the composition of the crystal form A of the compound represented by Formula I has excellent physicochemical stability.

1. A pharmaceutical composition comprising 5 wt. % to 90 wt. % of a compound represented by Formula I or a crystalline form thereof, or a mixture thereof,

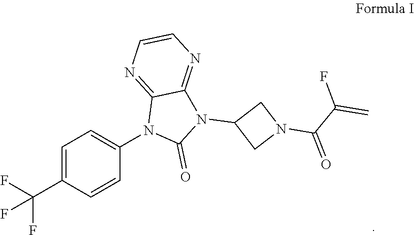 [SMILES: C=C(F)C(=O)N1CC(n2c(=O)n(-c3ccc(C(F)(F)F)cc3)c3nccnc32)C1]

2. The composition of claim 1, wherein the crystalline form of the compound represented by Formula I is a crystal form A, and the crystal form A has an X-ray powder diffraction pattern comprising characteristic peaks at 2θ values of 8.2°±0.2°, 15.4°±0.2°, and 18.2°±0.2°.

3. The composition of claim 2, wherein the crystal form A has an X-ray powder diffraction pattern comprising characteristic peaks at 2θ values of 6.4°±0.2°, 7.7°±0.2°, 8.2°±0.2°, 12.8°±0.2°, 15.4°±0.2°, and 18.2°±0.2°.

4. The composition of claim 2, wherein the crystal form A has an X-ray powder diffraction pattern comprising characteristic peaks at 2θ values of 6.4°±0.2°, 7.7°±0.2°, 8.2°±0.2°, 12.8°±0.2°, 15.4°±0.2°, 18.2°±0.2°, and 20.3°±0.2°.

5. The composition of claim 2, wherein the crystal form A has an X-ray powder diffraction pattern comprising characteristic peaks at 2θ values of 6.4°±0.2°, 7.7°±0.2°, 8.2°±0.2°, 12.8°±0.2°, 15.4°±0.2°, 18.2°±0.2°, 20.3°±0.2°, and 21.6°±0.2°.

6. The composition of claim 2, wherein the crystal form A has an X-ray powder diffraction pattern substantially as shown in FIG. 1.

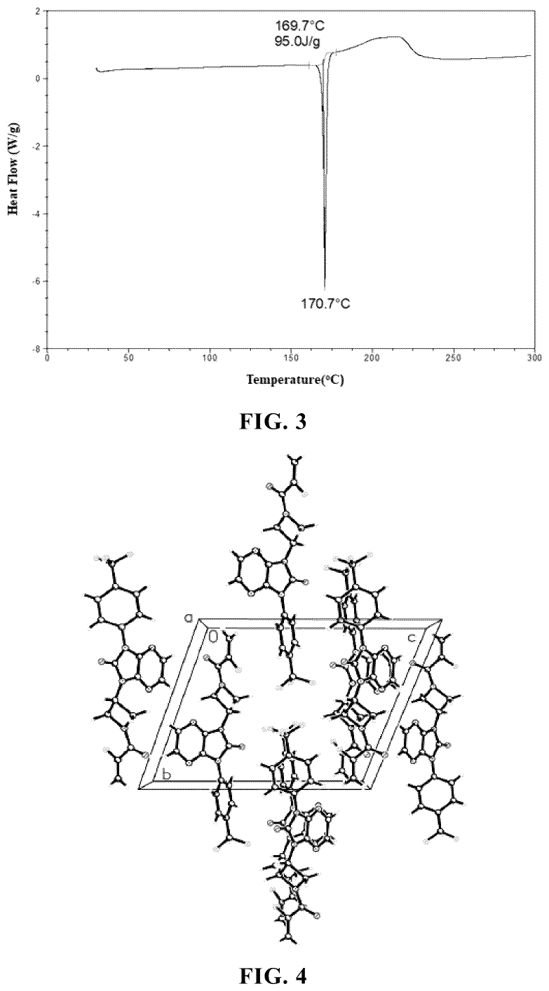

7. The composition of claim 2, wherein the crystal form A has a DSC pattern substantially as shown in FIG. 3.

8. The composition of claim 1, wherein the pharmaceutical composition further comprises one or more pharmaceutically acceptable carriers, and the pharmaceutically acceptable carriers comprise one or more of a diluent, a filler, a lubricant, a binder, and a disintegrating agent.

9. A crystal form A of a compound represented by Formula I, wherein the crystal form A has an X-ray powder diffraction pattern comprising characteristic peaks at 2θ values of 8.2°±0.2°, 15.4°±0.2°, and 18.2°±0.2°,

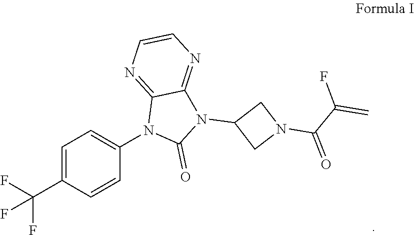 [SMILES: C=C(F)C(=O)N1CC(n2c(=O)n(-c3ccc(C(F)(F)F)cc3)c3nccnc32)C1]

10. The crystal form A of claim 9, wherein the crystal form A has an X-ray powder diffraction pattern comprising characteristic peaks at 2θ values of 6.4°±0.2°, 7.7°±0.2°, 8.2°±0.2°, 12.8°±0.2°, 15.4°±0.2°, and 18.2°±0.2°.

11. Use of the composition of claim 1 or the compound represented by Formula I or a crystalline form thereof, or a mixture thereof claim in the manufacture of a medicament, wherein the medicament is for treating a disease mediated by an abnormality of Hippo pathway.

12. The use of claim 11, wherein the disease is cancer, cancer metastasis, a proliferative disease or an inflammatory disease.

13. The use of claim 12, wherein the cancer is selected from the group consisting of colon cancer, stomach cancer, thyroid cancer, lung cancer, leukemia, pancreatic cancer, melanoma, multiple melanoma, brain cancer, kidney cancer, liver cancer, squamous cell carcinoma, gastrointestinal cancer, mesothelioma, skin cancer, prostate cancer, ovarian cancer, and breast cancer.

14. A method for treating a disease, comprising administering a therapeutically effective amount of the composition of claim 1 or the compound represented by Formula I or a crystalline form thereof, or a mixture thereof to a subject to be treated, wherein the disease is a disease mediated by an abnormality of Hippo pathway.

15. The method of claim 14, wherein the disease is cancer, a proliferative disease or an inflammatory disease, and wherein the cancer is selected from the group consisting of colon cancer, stomach cancer, thyroid cancer, lung cancer, leukemia, pancreatic cancer, melanoma, multiple melanoma, brain cancer, kidney cancer, liver cancer, squamous cell carcinoma, gastrointestinal cancer, mesothelioma, skin cancer, prostate cancer, ovarian cancer, and breast cancer.

16. A preparation method of a compound represented by Formula I, comprising the following steps: 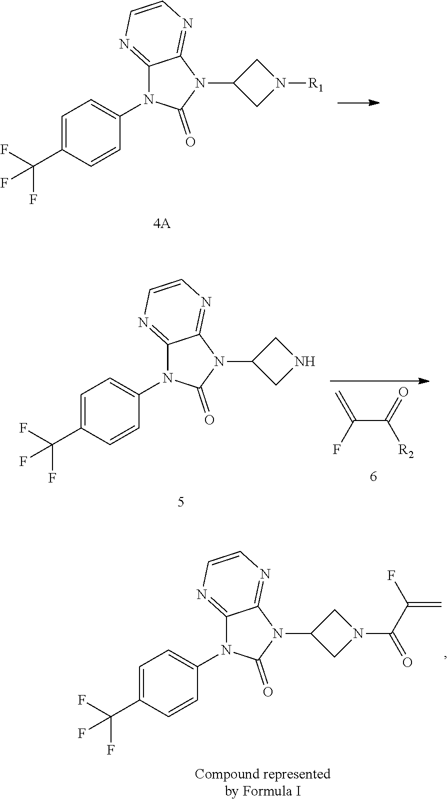
subjecting Compound 4A to a deprotection reaction to obtain Compound 5, and subjecting Compound 5 and Compound 6 to a substitution or condensation reaction to obtain the compound represented by Formula I;
wherein R₁ is a protecting group selected from the group consisting of —Boc, —Cbz, —Fmoc, trifluoroacetyl, —Trt, —PMB, and —Bn;
R₂ is selected from the group consisting of —Cl, imidazolyl, and —OR₃; and
R₃ is selected from the group consisting of H, —C₁₋₄ alkyl, phenyl, nitro-substituted phenyl, —C(O)—O—C₁₋₄ alkyl, —C(O)—O-phenyl, and —C(O)—O-nitro-substituted phenyl.

17. The preparation method of claim 16, wherein the method further comprises the following preparation steps of Compound 4A:
subjecting Compound A-1 and Compound A-2 to a coupling reaction under alkaline conditions to obtain Compound 4A; 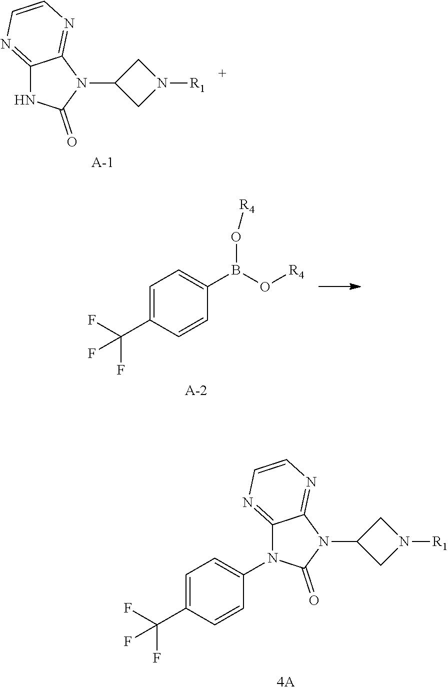
or
subjecting Compound B-1 to a ring-closing reaction to obtain Compound 4A, 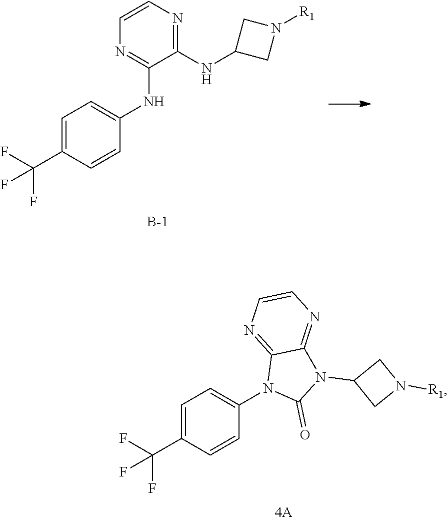
wherein R₁ is a protecting group selected from the group consisting of —Boc, —Cbz, —Fmoc, trifluoroacetyl, —Trt, —PMB, and —Bn; and
R₄ is selected from the group consisting of H and C₁₋₄ alkyl, or two R₄ and linked O atoms thereof together form 5-membered heterocycle containing two oxygen atoms and one boron atom, wherein the 5-membered heterocycle is optionally substituted with one or more C₁₋₄ alkyl.

18. The preparation method of claim 17, wherein the method further comprises the following preparation steps of Compound B-1:
subjecting Compound B-4 and Compound B-5 to a substitution reaction to obtain Compound B-2, 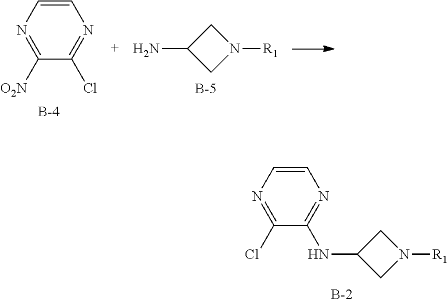
or
subjecting Compound B-6 and Compound B-5 to a substitution reaction to obtain Compound B-2, 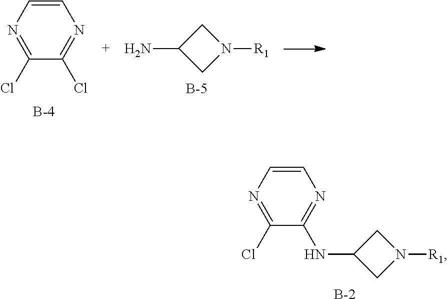
and then
subjecting Compound B-2 and Compound B-3 to a coupling reaction to obtain Compound B-1; 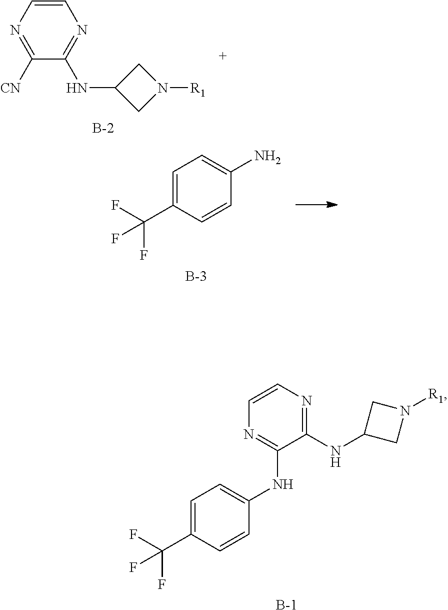
wherein R₁ is a protecting group selected from the group consisting of —Boc, —Cbz, —Fmoc, trifluoroacetyl, —Trt, —PMB, and —Bn.

19. An intermediate compound selected from the group consisting of 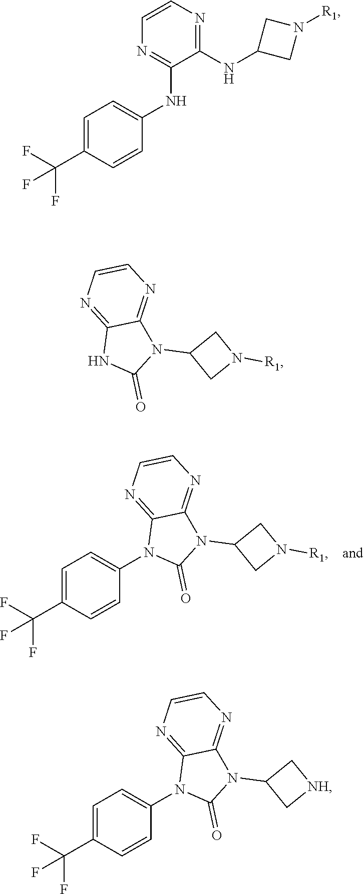
wherein R₁ is a protecting group selected from the group consisting of —Boc, —Cbz, —Fmoc, trifluoroacetyl, —Trt, —PMB, and —Bn.

20. The preparation method of claim 16, wherein R₁ is —Boc.
